(19) United States
(12) Patent Application Publication
(10) Pub. No.: US 2024/0270692 A1
(43) Pub. Date: Aug. 15, 2024

(54) S(+) FORM OF 4-(2,4-DIFLUOROPHENYL)-2-(1H-INDOL-3-YL)-4-OXO-BUTANOIC ACID

(71) Applicant: TOHOKU UNIVERSITY, Sendai-shi (JP)

(72) Inventors: Takaaki ABE, Miyagi (JP); Yotaro Matsumoto, Miyagi (JP); Takehiro Suzuki, Miyagi (JP); Ryota Kujirai, Miyagi (JP); Atsushi Higashitani, Miyagi (JP); Takeya Sato, Miyagi (JP); Eunsang Kwon, Miyagi (JP)

(73) Assignee: TOHOKU UNIVERSITY, Sendai-shi (JP)

(21) Appl. No.: 18/433,832

(22) Filed: Feb. 6, 2024

Related U.S. Application Data
(63) Continuation-in-part of application No. PCT/JP22/30040, filed on Aug. 5, 2022.
(63) Continuation-in-part of application No. PCT/JP23/36841, filed on Oct. 11, 2023.

(30) Foreign Application Priority Data
Aug. 6, 2021 (JP) 2021-130237

(51) Int. Cl. C07D 209/18; A61K 31/405; A61K 47/02; A61K 47/10; A61P 27/16

(52) U.S. Cl. CPC C07D 209/18; A61K 31/405; A61K 47/02; A61K 47/10; A61P 27/16

(57) ABSTRACT

An S(+) form of 4-(2,4-difluorophenyl)-2-(1H-indol-3-yl)-4-oxo-butanoic acid or a physiologically acceptable salt thereof may be useful as an active ingredient of a pharmaceutical composition or a food or drink composition, and the like. An enantiomeric excess of an S(+) form may be at least 97% or more. The compound may be used in treating, improving, or preventing a mitochondrial disease and/or a symptom or a disease associated with aging.

### CROSS-REFERENCE TO RELATED APPLICATIONS

The present application is a bypass continuing application based on and claiming priority to international application PCT/JP2022/030040, filed on Aug. 5, 2022, claiming the benefit of the filing date of Japanese Appl. No. 2021-130237, filed on Aug. 6, 2021, and to international application PCT/JP2023/036841, filed on Oct. 11, 2023, the content of each of which is incorporated by reference.

### TECHNICAL FIELD

The present invention relates to the enantiomer S(+) form of 4-(2,4-difluorophenyl)-2-(1H-indol-3-yl)-4-oxo-butanoic acid (hereinafter sometimes referred to as “MA5”) or a physiologically acceptable salt thereof and its use as an active ingredient of a pharmaceutical composition or a food or drink composition, and the like.

### BACKGROUND ART

The present inventors have reported that the racemic form of 4-(2,4-difluorophenyl)-2-(1H-indol-3-yl)-4-oxo-butanoic acid (that is, MA5) has an erythropoietin expression enhancing effect and a therapeutic effect on a mitochondrial disease (Patent Literature 1), an organ fibrosis suppressing effect (Patent Literature 2), and a preventive or improving effect on hearing loss (Patent Literature 3).

### CITATION LIST

### Patent Literature

[Patent Literature 1] International Publication No. WO 2014/080640
[Patent Literature 2] Japanese Patent Laid-Open No. 2015-189670
[Patent Literature 3] Japanese Patent Laid-Open No. 2019-116453

### SUMMARY OF INVENTION

The present inventors divided MA5 into its enantiomers and have confirmed that it is the S(+) form of MA5 that has the action of increasing the amount of NAD⁺ in an animal cell and has mitochondrial function activating and anti-aging actions.

The present invention relates to the S(+) form of MA5, and provides the following [1] to [12].
[1] A compound represented by the following formula (A-1), substantially free of an R(−) form, or a physiologically acceptable salt thereof.

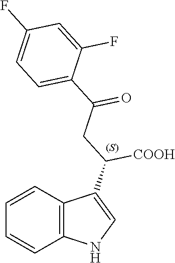

[2] The compound according to [1], wherein an enantiomeric excess of an S(+) form is at least 97% or more, or a physiologically acceptable salt thereof.
[3] A composition comprising the compound according to [1] or a physiologically acceptable salt thereof.
[4] The composition according to [3], wherein an enantiomeric excess of an S(+) form is at least 97% or more.
[5] The composition according to [3], which is stored with protection from light.
[6] The composition according to [3], which is stored near neutrality.
[7] The composition according to [3], which is stored at pH 6.0 to 8.0.
[8] The composition according to [3], when the composition comprises chloroform, a content of a short chain alcohol based on chloroform is less than 1%.
[9] The composition according to [3], which does not comprise a short chain alcohol and chloroform at the same time.
[10] The composition according to [3], which further comprises a physiologically acceptable carrier.
[11] The composition according to [3], which is a pharmaceutical composition.
[12] The composition according to [3], which is a food or drink composition.

In another embodiment, the present invention relates to a pharmaceutical composition, a food or drink composition, or the like including the S(+) form of MA5 as an active ingredient, and provides the following [1] to [15].
[1] A pharmaceutical composition comprising a compound represented by the following formula (A-1), substantially free of an R(−) form, or a physiologically acceptable salt thereof as an active ingredient.
[2] The pharmaceutical composition according to [1], wherein the pharmaceutical composition is used to increase an NAD⁺ level in a subject.
[3] The pharmaceutical composition according to [1] or [2], wherein the pharmaceutical composition is for treatment or prevention of a mitochondrial disease.
[4] The pharmaceutical composition according to [1] or [2], wherein the pharmaceutical composition is for treatment or prevention of a symptom or a disease associated with aging.
[5] The pharmaceutical composition according to [4], wherein the symptom or the disease associated with aging is hearing loss.
[6] The pharmaceutical composition according to any one of [1] to [5], wherein an enantiomeric excess of an S(+) form is at least 97% or more.
[7] A food or drink composition comprising a compound represented by the formula (A-1), substantially free of an R(−) form, or a physiologically acceptable salt thereof as an active ingredient.
[8] The food or drink composition according to [7], wherein the food or drink composition is used to increase an NAD⁺ level in a subject.
[9] The food or drink composition according to [7] or [8], wherein the food or drink composition is for a subject in need of treating or preventing a mitochondrial disease.
[10] The food or drink composition according to [7] or [8], wherein the food or drink composition is for a subject in need of treating or preventing a symptom or a disease associated with aging.
[11] The food or drink composition according to [10], wherein the symptom or the disease associated with aging is hearing loss.
[12] The food or drink composition according to any one of [7] to [11], wherein an enantiomeric excess of an S(+) form is at least 97% or more.
[13] The pharmaceutical composition according to any one of [1] to [6], or the food or drink composition according to any one of [7] to [12], wherein the pharmaceutical composition or the food or drink composition is stored with protection from light.
[14] The pharmaceutical composition according to any one of [1] to [6], or the food or drink composition according to any one of [7] to [12], wherein the pharmaceutical composition or the food or drink composition is stored near neutrality, preferably at pH 6.0 to 8.0.
[15] The pharmaceutical composition according to any one of [1] to [6], or the food or drink composition according to any one of [7] to [12], wherein when the pharmaceutical composition or the food or drink composition comprises chloroform, a content of a short chain alcohol based on chloroform is less than 1%, and the pharmaceutical composition or the food or drink composition preferably does not comprise a short chain alcohol and chloroform at the same time.

In another embodiment, the present invention provides the following [1] to [5] (the compound represented by formula (A-1) is as described above).
[1] A method for increasing an NAD⁺ level in a subject, comprising administering a compound represented by formula (A-1), substantially free of an R(−) form, or a physiologically acceptable salt thereof to the subject in need thereof.
[2] A method for treating, improving, or preventing a mitochondrial disease, comprising administering a compound represented by formula (A-1), substantially free of an R(−) form, or a physiologically acceptable salt thereof to a subject in need thereof.
[3] A method for treating, improving, or preventing a symptom or a disease associated with aging, comprising administering a compound represented by formula (A-1), substantially free of an R(−) form, or a physiologically acceptable salt thereof to a subject in need thereof.
[4] The method according to [3], wherein the symptom or the disease associated with aging is any selected from the group consisting of visual impairment, anemia, muscle weakness, thinning hair or hair loss, and shortened lifespan.
[5] The method according to [3], wherein the symptom or the disease associated with aging is hearing loss.

In addition, other embodiments of the present invention include:
use of an S(+) form of MA5 or a physiologically acceptable salt thereof in production of a therapeutic agent or a preventive agent for a mitochondrial disease; use of an S(+) form of MA5 or a physiologically acceptable salt thereof in production of a therapeutic agent or a preventive agent for a symptom or a disease associated with aging;
a method for promoting generation of NAD⁺ in an animal (subject) cell, comprising a step of administering an S(+) form of MA5 or a physiologically acceptable salt thereof to a subject in need of promoting generation of NAD⁺ in an animal cell;
a method for in vitro promoting generation of NAD⁺ in an animal cell, comprising a step of culturing an animal cell in the presence of an S(+) form of MA5 or a physiologically acceptable salt thereof (hereinafter sometimes referred to as “the present promotion method”); a mitochondrial function activating agent in an animal, comprising an S(+) form of MA5 or a physiologically acceptable salt thereof;
a method for activating a mitochondrial function in an animal, comprising a step of administering an S(+) form of MA5 or a physiologically acceptable salt thereof to a subject in need of activating the mitochondrial function; an agent for improving motor dysfunction in an animal, comprising an S(+) form of MA5 or a physiologically acceptable salt thereof; and
a method for improving motor dysfunction in an animal, comprising a step of administering an S(+) form of MA5 or a physiologically acceptable salt thereof to a subject in need of improving the motor dysfunction.

### Advantageous Effects of Invention

Unlike the racemic form of MA5 and the R(−) form of MA5, the S(+) form of MA5, which is a compound of the present invention, has the action of increasing the amount of NAD⁺ in an animal cell. In addition, unlike the R(−) form of MA5, the S(+) form of MA5 has the action of increasing the expression level of an SIRT in an animal cell. Furthermore, the S(+) form of MA5 is less likely to be metabolized into a toxic substance (glucuronide conjugate) in vivo, is more likely to be retained in blood, and has lower cytotoxicity to an animal cell than the R(−) form of MA5.

On the other hand, it is known that the NAD⁺ level in an animal cell decreases with age and plays an important role in the onset of an age-related disease. In addition, it has been reported that increasing the amount of NAD⁺ extends the lifespan in C. elegans and provides a protective action against a metabolic disease in a mouse (see the literature “Nature. 2018 563(7731) 354-359.”). In addition, C. elegans is used as a model organism for human aging (see the literature “Bulletin of the Institute of Advanced Biosciences, Tokai University, Volume 1, March 2017”).

Because of these, when the S(+) form of MA5 is administered to an animal, it increases the amount of NAD⁺ in an animal cell without causing any adverse effect such as a side effect, and as a result, it can be fully expected that the lifespan can be extended, or a symptom or a disease such as a symptom or a disease associated with aging or a metabolic disease can be treated or prevented.

Furthermore, the S(+) form of MA5 has the action of improving a mitochondrial function in a cell, and thus is useful as a mitochondrial function activating agent or a therapeutic agent or a preventive agent for a mitochondrial disease.

### BRIEF DESCRIPTION OF DRAWINGS

FIG. 1 is a diagram showing results obtained by culturing a rat pancreatic β cell line (INS-1E cell line) for 6 hours in the presence of 0.1% DMSO (“0” in the figure) or various concentrations (3 pM, 10 pM, 30 pM, 100 pM, 300 pM, 1 nM, or 3 nM) of three compounds (racemic form of MA5 [FIG. 1A], S(+) form of MA5 [FIG. 1B], or R(−) form of MA5 [FIG. 1C]), and measuring the amount of NAD⁺ in cells (average value±standard error [SE]). “*” and “**” in the figure indicate a statistically significant increase (p<0.05 and p<0.01, respectively) as compared with the results in the absence of the compounds, and “#” and “##” in the figure indicate a statistically significant decrease (p<0.05 and p<0.01, respectively) as compared with the results with 0.1% DMSO.

FIG. 2 is a diagram showing results obtained by incubating human liver microsomes in the presence of three compounds (racemic form of MA5 [“racemi” in the figure], S(+) form of MA5 [“S” in the figure], or R(−) form of MA5 [“R” in the figure] or in the absence of these compounds (“Control” in the figure) and analyzing the generated metabolites by mass spectrometry.

FIG. 3 is a diagram showing results obtained by culturing a human bladder epithelial-derived cell line (HBlEpC cell line) in the presence of 0.1% DMSO (“0” in the figure) or various concentrations (30 nM, 100 nM, 300 nM, 1 μM, 3 μM, 10 μM, 30 μM, 100 μM, 300 μM, or 1 mM) of three compounds (racemic form of MA5 [FIG. 3A], S(+) form of MA5 [FIG. 3B], or R(−) form of MA5 [FIG. 3C]) and analyzing the viable cell level (average value±SE). The “viable cell level” on the vertical axis indicates the value of optical density (OD₄₅₀) at a wavelength of 450 nm measured by a WST-8 assay. “*” and “**” in the figure indicate that there is a statistically significant difference (p<0.05 and p<0.01, respectively) as compared with the results with 0.1% DMSO.

FIG. 4 is a diagram showing results obtained by culturing a fibroblast cell line derived from a Leigh encephalopathy patient (MT-ND3 mutant cell line) in the presence of 0.1% DMSO (“0” in the figure) or various concentrations (0.003 μM, 0.01 μM, 0.03 μM, 0.1 μM, 0.3 M, 1 μM, 3 μM, 10 μM, 30 μM, or 100 μM) of each enantiomer of MA5 (S (+) form [“S” in the figure] or R(−) form [“R” in the figure]) and measuring the viable cell level (average value±SE). “*” in the figure indicates that there is a statistically significant difference (p<0.05) as compared with the results with 0.1% DMSO.

FIG. 5 is a diagram showing results obtained by culturing the MT-ND3 mutant cell line in the presence of 0.1% DMSO (“0” in the figure) or various concentrations (0.1 μM or 1 μM) of three compounds (racemic form of MA5 [“racemi” in the figure], S(+) form of MA5 [“S” in the figure], or R(−) form of MA5 [“R” in the figure]) and measuring the amount of ATP produced in the culture medium. “*” in the figure indicates that there is a statistically significant difference (p<0.05) as compared with the results with 0.1% DMSO.

FIG. 6 is fluorescence images of mitochondria in body wall muscle cells of ccIs4251 wild-type C. elegans (“WT” in the figure; n=18) or immt-1/mitofilin mutants (“immt-1/mitofilin mutant mock”; n=41, “immt-1/mitofilin mutant MA-5 (racemi)”; n=37, “immt-1/mitofilin mutant MA-5S”; n=61, and “immt-1/mitofilin mutant MA-5R”; n=34) raised in the presence of four substances (DMSO or 10 μM racemic form of MA5, S(+) form of MA5, or R(−) form of MA5, respectively).

FIG. 7 is a diagram showing results obtained by analyzing mitochondrial swelling levels in body wall muscle cells based on the fluorescence images in FIG. 6.

FIG. 8 is a diagram showing results obtained by analyzing mitochondrial swelling levels in body wall muscle cells based on the fluorescence images in FIG. 6. It is indicated that there is a statistically significant difference (p<0.05) between “a” and “b” in the figure. “x” in the figure indicates an average value, and “◯” in the figure indicates an outlier.

FIG. 9 is a diagram showing results obtained by orally administering the S(+) form of MA5 and the R(−) form of MA5 to cynomolgus monkeys, respectively, and measuring the concentrations of the S(+) form of MA5 and the R(−) form of MA5 in the plasma after administration.

FIG. 10 is a diagram showing results obtained by orally administering the S(+) form of MA5 and the R(−) form of MA5 to cynomolgus monkeys, respectively, and measuring the concentrations of a reduced metabolite of MA5 (compound represented by formula (B) [hereinafter also referred to as “Red-MA5” ]) in the plasma after administration.

FIG. 11 is a diagram showing results obtained by culturing a fibroblast cell line derived from a Leigh encephalopathy patient (KCMC10 cell line) in the presence of 0.1% DMSO (“0” in the figure) or various concentrations (3 nM, 10 nM, 30 nM, 100 nM, 300 nM, 1 μM, 3 μM, 10 μM, or 30 μM) of two compounds (racemic form of MA5 [FIG. 11A] or Red-MA5 [FIG. 11B]) and measuring the viable cell level (average value±SE). “*” in the figure indicates that there is a statistically significant difference (p<0.05) as compared with the results with 0.1% DMSO.

FIG. 12-1 is a diagram showing results obtained by culturing a mouse inner ear cell line (HEI-OC1 cell line) in the presence of 0.1% DMSO or various concentrations (1 M, 3 μM, 10 μM, or 30 μM) of the racemic form of MA5, and analyzing the expression levels of various SIRTs (SIRT1, SIRT2 isoform 1, SIRT2 isoform 2, and SIRT3) in the cells by Western blotting. The lower graphs show results obtained by expressing each band intensity in the upper results as a relative value when the band intensity in 0.1% DMSO, which is the control, is set to 1.

FIG. 12-2 is a diagram showing results obtained by culturing the HEI-OC1 cell line in the presence of 0.1% DMSO or various concentrations (1 μM, 3 μM, 10 μM, or 30 M) of the racemic form of MA5, and analyzing the expression levels of various SIRTs (SIRT5, SIRT6, and SIRT7) and the expression level of 0-actin (FIG. 13D) in the cells by Western blotting. The lower graphs show results obtained by expressing each band intensity in the upper results as a relative value when the band intensity in 0.1% DMSO, which is the control, is set to 1.

FIG. 13-1 is a diagram showing results obtained by culturing the HEI-OC1 cell line in the presence of 0.1% DMSO or various concentrations (1 μM, 3 μM, 10 μM, or 30 M) of the S(+) form of MA5, and analyzing the expression levels of various SIRTs (SIRT1, SIRT2 isoform 1, SIRT2 isoform 2, and SIRT3) in the cells by Western blotting. The lower graphs show results obtained by expressing each band intensity in the upper results as a relative value when the band intensity in 0.1% DMSO, which is the control, is set to 1.

FIG. 13-2 is a diagram showing results obtained by culturing the HEI-OC1 cell line in the presence of 0.1% DMSO or various concentrations (1 μM, 3 μM, 10 μM, or 30 M) of the S(+) form of MA5, and analyzing the expression levels of various SIRTs (SIRT5, SIRT6, and SIRT7) and the expression level of β-actin in the cells by Western blotting. The lower graphs show results obtained by expressing each band intensity in the upper results as a relative value when the band intensity in 0.1% DMSO, which is the control, is set to 1.

FIG. 14-1 is a diagram showing results obtained by culturing the HEI-OC1 cell line in the presence of 0.1% DMSO or various concentrations (1 μM, 3 μM, 10 μM, or 30 M) of the R(−) form of MA5, and analyzing the expression levels of various SIRTs (SIRT1, SIRT2 isoform 1, SIRT2 isoform 2, and SIRT3) in the cells by Western blotting. The lower graphs show results obtained by expressing each band intensity in the upper results as a relative value when the band intensity in 0.1% DMSO, which is the control, is set to 1.

FIG. 14-2 is a diagram showing results obtained by culturing the HEI-OC1 cell line in the presence of 0.1% DMSO or various concentrations (1 μM, 3 μM, 10 μM, or 30 M) of the R(−) form of MA5, and analyzing the expression levels of various SIRTs (SIRT5, SIRT6, and SIRT7) and the expression level of β-actin in the cells by Western blotting. The lower graphs show results obtained by expressing each band intensity in the upper results as a relative value when the band intensity in 0.1% DMSO, which is the control, is set to 1.

FIG. 15 shows results of a test of binding of the S(+) form and the R(−) form of MA5 to NAMPT. The Kd value of the S(+) form of MA5 was 58.94 μM, whereas the R(−) form of MA5 did not bind to NAMPT, and the Kd value was unable to be measured.

FIG. 16 shows the fluorescence intensity by immunostaining of γH2AX (phosphorylated H2AX). Only when the S(+) form of MA5 was added, the level of γH2AX was low, and a suppressing action on DNA damage was observed.

FIG. 17 shows chromatograms of MA5 using a preparative separation apparatus (LC-Forte). As a result of X-ray crystallography of the corresponding benzylamine salts, it was determined that the absolute configuration of MA5 in the pre-elution fraction was S, and that the absolute configuration of MA5 in the post-elution fraction was R. In addition, as a result of measuring the optical rotation of MA5, it was determined that MA5 in the pre-elution fraction was dextrorotatory (+), and that MA5 in the post-elution fraction was levorotatory (−).

### DESCRIPTION OF EMBODIMENTS

The compound of the present invention is the enantiomer S(+) form of the compound represented by the following formula (A-1) (that is, the compound represented by the following formula (A) (4-(2,4-difluorophenyl)-2-(1H-indol-3-yl)-4-oxo-butanoic acid (MA5)) or a physiologically acceptable salt thereof.

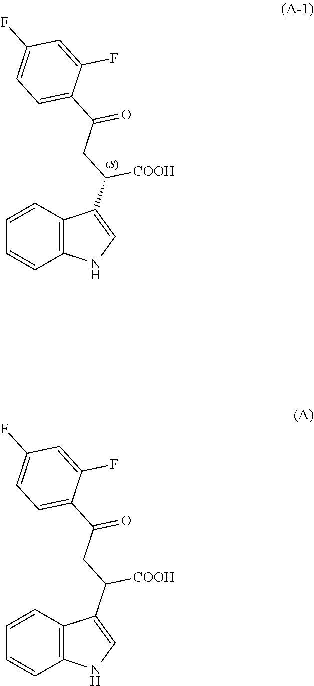

The S(+) form of MA5 used in the present invention is substantially free of the R(−) form of MA5. “Substantially free of the R(−) form of MA5” means that the enantiomeric excess of the S(+) form is about 97% or more, preferably about 97.5% or more, more preferably about 98% or more, particularly preferably about 98.5% or more, and further preferably about 99% or more, for example, about 99.5%, 99.6%, 99.7%, 99.8%, or 99.9% or more.

The “enantiomeric excess” (also written as “ee”) refers to the excess of one enantiomer over the other enantiomer, and when the S form is in excess, the enantiomeric excess is expressed by the following expression.

$$ee = ( {A}_{S} - {A}_{R} ) / ( {A}_{S} - {A}_{R} )$$

A_{R} and A_{S} represent the molar fractions of the R form and the S form, respectively.

As used herein, “about” refers to a value that may vary up to plus or minus 10%, 8%, 6%, 5%, 4%, 3%, 2%, or 1% from the reference value. Preferably, the term “about” refers to a range of plus or minus 10%, 5%, or 1% from the reference value.

As used herein, “physiologically acceptable salt” is a salt that, within the scope of an appropriate medical, pharmaceutical, or biological determination, is commensurate with a moderate benefit-risk ratio without causing undue toxicity, irritation, allergic response, or a further problem or complication for use in contact with an animal tissue. Examples of the physiologically acceptable salt include an ammonium salt; an alkali metal salt such as a sodium salt, a lithium salt, or a potassium salt; an alkaline earth metal salt such as an aluminium salt, a calcium salt, or a magnesium salt; a salt with an organic base such as a dicyclohexylamine salt or N-methyl-D-glucamine; a salt with an amino acid such as arginine, lysine, or ornithine; and a salt generated by a basic nitrogen-containing group.

The compound represented by formula (A-1) in the present invention can be obtained according to an organic synthesis method using a known organic chemical reaction, for example, the method described in the section “1. Generation of S(+) form of MA5” in the Examples described below.

The present invention provides a pharmaceutical composition or a food or drink composition including the S(+) form of MA5 or a physiologically acceptable salt thereof as an active ingredient. The pharmaceutical composition or the food or drink composition is substantially free of the R(−) form of MA5 or a physiologically acceptable salt thereof. “Substantially free of the R(−) form of MA5 or a physiologically acceptable salt thereof” means that the enantiomeric excess of the S(+) form is about 97% or more, preferably about 97.5% or more, more preferably about 98% or more, particularly preferably about 98.5% or more, and further preferably about 99% or more, for example, about 99.5%, 99.6%, 99.7%, 99.8%, or 99.9% or more.

In one embodiment, the pharmaceutical composition or the food or drink composition is used to promote generation of NAD⁺ in an animal cell, and for example, to increase the NAD⁺ level in a subject in need thereof.

In another embodiment, the pharmaceutical composition or the food or drink composition is used to activate a mitochondrial function in a subject, and for example, to treat or prevent a mitochondrial disease in a subject in need thereof.

In another embodiment, the pharmaceutical composition or the food or drink composition is used to treat or prevent a symptom or a disease associated with aging in a subject in need thereof.

Examples of the food or drink composition include a health food (functional food, nutritional supplement, dietary supplement, fortified food, nutrient controlling food, supplement, or the like) and a food with health claims (food for special dietary uses, food with nutrient function claims, food with function claims, or the like).

Herein, examples of the animal include a mammal (human or non-human mammal), a bird, a reptile, an amphibian, a fish, and an invertebrate. In addition, other embodiments of the animal include a human and a domestic animal. Here, the “domestic animal” means an animal that is raised and bred by a human. Examples of the domestic animal include a non-human mammalian animal (for example, a rodent such as a mouse, a rat, a hamster, or a guinea pig; a lagomorph such as a rabbit; an ungulate such as a pig, a cow, a goat, a horse, or a sheep; a carnivore such as a dog or a cat), a bird (for example, a chicken, a quail, a turkey, a pigeon, a duck, or a goose), a fish (for example, a carp or a goldfish), and an invertebrate (for example, a silkworm or a honeybee).

Herein, the pharmaceutical composition or the food or drink composition may include an additive. Examples of the additive include a physiologically acceptable common blending component such as a carrier, a binder, a stabilizer, an excipient, a diluent, a pH buffering agent, a disintegrant, an isotonic agent, a coating agent, a solubilizer, a lubricating agent, a gliding agent, a dissolution enhancer, a lubricant, a flavoring agent, a sweetener, a solvent, a gelling agent, or a nutrient. Specific examples of the blending component include water, physiological saline, an animal fat and oil, a vegetable oil, lactose, starch, gelatin, crystalline cellulose, gum, talc, magnesium stearate, hydroxypropyl cellulose, polyalkylene glycol, polyvinyl alcohol, and glycerin.

The S(+) form of MA5 exhibits a physical property different from that of the racemic form, and is particularly easily colored and degraded under a light, acid, or alkali condition. Therefore, the pharmaceutical composition or the food or drink composition of the present invention is preferably stored with protection from light. In addition, the pharmaceutical composition or the food or drink composition of the present invention is preferably stored near neutrality, preferably at pH 6.0 to 8.0. In addition, the S(+) form of MA5 is easily degraded in the presence of a short chain alcohol and chloroform, and thus the pharmaceutical composition or the food or drink composition of the present invention does not include a short chain alcohol and chloroform at the same time, and particularly when the pharmaceutical composition or the food or drink composition includes chloroform, the content of the short chain alcohol based on chloroform is preferably less than 1%.

Herein, the “mitochondrial disease” may be a symptom caused by a decrease in a mitochondrial function such as ATP production, regulation of apoptosis, and regulation of the intracellular concentration of a calcium ion or iron due to a genetic mutation in cell nuclear DNA or mitochondrial DNA or the like, and specific examples thereof include CPEO (chronic progressive external ophthalmoplegia syndrome), MELAS (encephalomyopathy, lactic acidosis, and stroke-like syndrome), MERRF (myoclonic epilepsy with ragged red fibers syndrome), Leigh encephalopathy (subacute necrotizing encephalomyelopathy), Leber disease (Leber hereditary optic neuropathy [LHON]), Kearns-Sayre syndrome (KSS), Barth syndrome, Pearson disease, and Friedreich ataxia (FRDA).

Herein, examples of “the symptom or the disease associated with aging” include thinning hair or hair loss (more specifically, age-related thinning hair or hair loss), hearing loss (more specifically, age-related hearing loss), visual impairment (more specifically, age-related visual impairment), anemia (more specifically, age-related anemia), muscle weakness (more specifically, age-related muscle weakness), and shortened lifespan (more specifically, age-related shortened lifespan).

Herein, examples of the “subject in need of increasing an NAD⁺ level” or the “subject in need of promoting generation of NAD⁺” include a subject in need of extending the lifespan (subject in need of longevity); a patient with a neurological disease (Parkinson's disease, depression, Alzheimer disease, amyotrophic lateral sclerosis [ALS], or the like); a patient with a cardiovascular disease (heart failure, arrhythmia, or the like); a patient with a muscle disease (sarcopenia, inclusion body myositis, muscular dystrophy, or the like); a patient with a renal disease (chronic kidney disease, renal failure, diabetic nephropathy, nephritis, or the like); a patient with a metabolic disease (diabetes, liver dysfunction, alcoholic liver disorder, non-alcoholic fatty liver disease [NAFLD], non-alcoholic steatohepatitis [NASH], thyroid/adrenal gland disease, or the like); a patient with a digestive system disease (inflammatory bowel disease, or the like); a cancer patient; a patient with a symptom or a disease associated with aging (thinning hair or hair loss, hearing loss, visual impairment, anemia, muscle weakness, shortened lifespan, or the like); and a patient with radiation injury, and the pharmaceutical composition or the food or drink composition of the present invention is administered to or ingested by those subjects. The pharmaceutical composition or the food or drink composition of the present invention can increase the NAD⁺ level (promote generation of NAD⁺) in cells of those subjects and as a result extend the lifespan or prevent or treat the above diseases and disorders.

Herein, examples of the “subject in need of activating a mitochondrial function” include a patient suffering from a disease related to or caused by a diminished or impaired mitochondrial function, and specific examples thereof include a patient suffering from diabetes, a mitochondrial disease, a brain disease, or the like.

Herein, the administration form of the pharmaceutical composition or the food or drink composition is not particularly limited, and examples thereof include oral administration by administration in a dosage form such as a powder, a granule, a tablet, a capsule, a syrup, or a suspension, and parenteral administration by injection (for example, subcutaneous injection, intravenous injection, or intramuscular injection) in a dosage form such as a solution, an emulsion, or a suspension or by intranasal administration in the form of a spray.

Herein, the dosage (intake) of the S(+) form of MA5 or a physiologically acceptable salt thereof in the pharmaceutical composition or the food or drink composition is appropriately determined depending on the age, the body weight, the sex, the symptom, the sensitivity to an agent, and the like, and is, for example, in the range of 1 μg to 200 mg/kg (body weight)/day. The pharmaceutical composition or the food or drink composition can be administered in a single dose or a plurality of (for example, 2 to 4) divided doses per day.

The present description provides a method for in vitro promoting generation of NAD⁺ in an animal cell, including culturing an animal cell in the presence of an S(+) form of MA5 or a physiologically acceptable salt thereof. The culture temperature of the animal cell in the above method is usually in the range of 30 to 40° C., and preferably about 37° C. (36 to 38° C.). In addition, the CO₂ concentration during culture is usually in the range of about 1 to 10%, and preferably about 5% (4 to 6%). In addition, the O₂ concentration during culture is usually in the range of about 10 to 40%, and preferably about 21% (20 to 22%).

In the above method, examples of the culture medium used when culturing the animal cell include a serum-containing or serum-free culture medium, physiological saline, phosphate-buffered physiological saline, Tris-buffered physiological saline, HEPES-buffered physiological saline, Ringer's solution (lactate Ringer's solution, acetate Ringer's solution, bicarbonate Ringer's solution, or the like), a physiological aqueous solution such as a 5% glucose aqueous solution; and here, examples of the serum-containing culture medium include a culture medium for mammalian cell culture (DMEM, EMEM, IMDM, RPMI-1640, αMEM, F-12, F-10, M-199, AIM-V, or the like) containing 0.1 to 30 (v/v) % serum (fetal bovine serum (FBS), calf bovine serum (CS), or the like), and examples of the serum-free culture medium include the above culture medium for mammalian cell culture supplemented with an appropriate amount (for example, 1 to 30%) of a serum substitute such as commercially available B27 Supplement (-Insulin) (manufactured by Life Technologies Corporation), N2 Supplement (manufactured by Life Technologies Corporation), B27 Supplement (manufactured by Life Technologies Corporation), or Knockout Serum Replacement (manufactured by Invitrogen Corporation).

The present invention provides a method for increasing an NAD⁺ level in a subject, including administering an S(+) form of MA5 or a physiologically acceptable salt thereof to the subject, wherein the S(+) form of MA5 or the physiologically acceptable salt thereof is substantially free of an R(−) form of MA5 or a physiologically acceptable salt thereof.

In another embodiment, the present invention provides a method for treating or preventing a mitochondrial disease in a subject, including administering an S(+) form of MA5 or a physiologically acceptable salt thereof to the subject, wherein the S(+) form of MA5 or the physiologically acceptable salt thereof is substantially free of an R(−) form of MA5 or a physiologically acceptable salt thereof.

In another embodiment, the present invention provides a method for treating or preventing a symptom or a disease associated with aging, including administering an S(+) form of MA5 or a physiologically acceptable salt thereof to a subject, wherein the S(+) form of MA5 or the physiologically acceptable salt thereof is substantially free of an R(−) form of MA5 or a physiologically acceptable salt thereof.

“Substantially free of an R(−) form of MA5 or a physiologically acceptable salt thereof” means that the enantiomeric excess of the S(+) form is about 97% or more, preferably about 97.5% or more, more preferably about 98% or more, particularly preferably about 98.5% or more, and further preferably about 99% or more, for example, about 99.5%, 99.6%, 99.7%, 99.8%, or 99.9% or more.

Hereinafter, the present invention will be described more specifically with reference to Examples, but the technical scope of the present invention is not limited to these Examples. The INS-1E cell line was cultured in an incubator (37° C., 21% O₂, 5% CO₂) in the presence of RPMI-1640 Medium (manufactured by GIBCO) containing 11.1 mM glucose, 10% FBS, 1 mM sodium pyruvate, 10 mM HEPES, 2 mM glutamine, 50 μM β-mercaptoethanol, penicillin at 100 U/mL, and streptomycin at 100 μg/mL (hereinafter simply referred to as “RPMI-1640 Medium”). In addition, the fibroblast cell line derived from a Leigh encephalopathy patient (MT-ND3 mutant cell line [Leigh synd., m.10191 T>C [p.Ser45Pro]]) was incubated in the above incubator in the presence of DMEM Low Glucose (manufactured by GIBCO) culture medium containing 1% FBS, penicillin at 100 U/mL, and streptomycin at 100 μg/mL (hereinafter referred to as “DMEM Low Glucose culture medium”). In addition, the HBlEpC cell line was incubated in the above incubator in the presence of Human Bladder Epithelial Cell Basal medium (manufactured by Cell Applications, Inc.) culture medium containing 10% FBS, penicillin at 100 U/mL, streptomycin at 100 μg/mL, and HBlEpC Growth Supplement (manufactured by Cell Application) (hereinafter referred to as “HBlEpC culture medium”).

### Examples

### 1. Generation of S(+) Form of MA5

### 1-1 Generation of trans-4-(2,4-difluorophenyl)-4-oxo-2-butenoic Acid

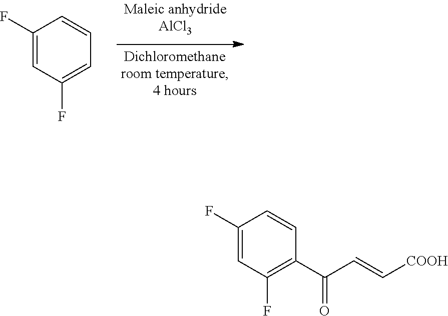 [SMILES: Fc1cccc(F)c1 ; O=C(O)/C=C/C(=O)c1ccc(F)cc1F]

In a 50 mL round bottom flask filled with nitrogen, 1,3-difluorobenzene (0.51 g, 4.47 mmol) was dissolved in dichloromethane (20 mL), maleic anhydride (0.43 g, 4.46 mmol) and aluminum chloride (1.20 g, 9.01 mmol) were added, and the resulting mixture was stirred at room temperature for 4 hours. The reaction solution was adjusted to pH 1 by adding 1 N hydrochloric acid (10 mL), and extracted with ethyl acetate (40 mL) three times. The organic layer was washed with saturated saline and dried over anhydrous sodium sulfate. The solvent was distilled off under reduced pressure, and then the residue was purified by recrystallization with benzene to obtain trans-4-(2,4-difluorophenyl)-4-oxo-2-butenoic acid. (0.57 g, yield 56%): melting point 114.8-119.6° C.; 1H NMR acetone-d₆): δ 7.98 (m, 1H), 7.71 (dd, J_{H-F}=15.6, 3.4 Hz, 1H), 7.23 (m, 2H), 6.75 (dd, J_{H-F}=15.6, 1.2 Hz, 1H); ¹³C NMR (acetone-d₆): δ 187.2 (d, J_{C-F}=2.6 Hz), 166.9 (dd, J_{C-F}=254.5, 12.3 Hz), 166.4, 163.4 (dd, J_{C-F}=254.5, 12.9 Hz), 140.0 (d, J_{C-F}=6.1 Hz), 134.0 (dd, J_{C-F}=10.9, 3.6 Hz), 133.0 (d, J_{C-F}=1.6 Hz), 123.3 (dd, J_{C-F}=12.4, 3.6 Hz), 113.4 (dd, J_{C-F}=21.5, 3.6 Hz), 105.8 (dd, J_{C-F}=27.3, 26.3 Hz); IR (neat): 2917, 1697, 1661 cm-1; FAB-MS m/z [M+H⁺] calcd for 213 (C₁₁H₁₀O₃), found 213.

### 1-2 Generation of Racemic Form of MA5

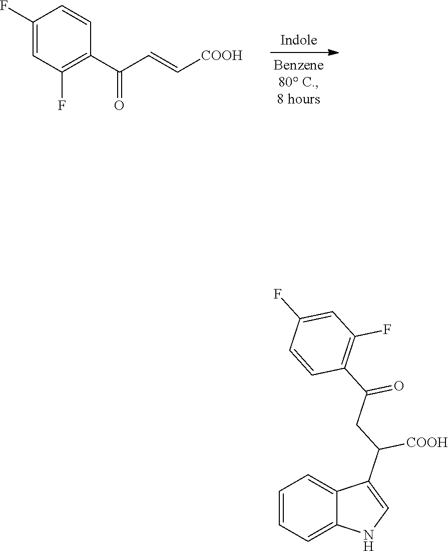 [SMILES: O=C(O)/C=C/C(=O)c1ccc(F)cc1F ; O=C(CC(C(=O)O)c1c[nH]c2ccccc12)c1ccc(F)cc1F]

In a 30 mL round bottom flask, trans-4-(2,4-difluorophenyl)-4-oxo-2-butenoic acid (0.39 g, 1.84 mmol) was dissolved in benzene (10 mL), and indole (0.26 g, 2.19 mmol) was added, and the resulting mixture was stirred at 80° C. for 8 hours and stirred until the temperature reached room temperature. The reaction solution was distilled off under reduced pressure, and the residue was purified by using silica gel column chromatography (chloroform:methanol=20:1) to obtain 4-(2,4-difluorophenyl)-2-(1H-indol-3-yl)-4-oxo-butanoic acid (racemic form of MA5) (see Table 3). (0.30 g, yield 51%): melting point 180.2-184.6° C.; ¹H NMR (DMSO-d₆): δ 7.98 (m, 1H), 7.65 (d, J=7.9 Hz, 1H), 7.37 (d, J=8.1 Hz, 1H), 7.42 (m, 1H), 7.28 (d, J=2.3 Hz, 1H), 7.24 (m, 1H), 7.09 (t, J=7.1 Hz, 1H), 7.01 (t, J=7.5 Hz, 1H), 4.34 (dd, J=10.5, 3.5 Hz, 1H), 3.90 (ddd, J_{H-F}=18.5, 10.6, 2.4 Hz, 1H), 3.30 (ddd, J_{H-F}=18.5, 6.1, 3.5 Hz, 1H); ¹³C NMR (DMSO-d₆): δ 195.2 (d, J_{C-F}=4.1 Hz), 174.8, 165.2 (d, J_{C-F}=253.0, 13.4 Hz), 162.2 (d, J_{C-F}=255.5, 13.4 Hz), 136.4, 132.7 (dd, J_{C-F}=10.8, 4.1 Hz), 126.3, 123.3, 122.2 (dd, J_{C-F}=12 3, 3.6 Hz), 121.4, 119.1, 118.8, 112.6 (dd, J_{C-F}=21.1, 3.6 Hz), 111.9, 111.8, 105.4 (dd, J_{C-F}=26.1 Hz), 45.6 (d, J_{C-F}=6.3 Hz), 37.9; IR (neat): 3382, 2919, 1678 cm-1; HRFAB m/z [M+H]⁺ calcd for 332.1036 (C₁₉H₁₇NO₃), found 312.1028.

### 1-3 Preparative Separation of S(+) Form of MA5

Preparative separation of the S(+) form of MA5 from the racemic form of MA5 synthesized was carried out by using preparative separation apparatus LC-Forte (manufactured by YMC Co., Ltd.). In addition, as a preparative column, CHIRALPAK AD-H (column inner diameter of 2 cm, column length of 25 cm, manufactured by Daicel Corporation) was used. The S(+) form was eluted over 40 minutes at a flow rate of 10 mL/min by using solution A (0.05% TFA/hexane), solution B (0.05% TFA/2-propanol), and a mobile phase having a single composition of solution A/solution B=60/40. The flow rate of the mobile phase can also be 20 mL/min, and the elution time can also be 20 minutes, which is half of the above time. The detection wavelength for MA5 was set to 220 nm. The retention times of the S(+) form and the R(−) form of MA5 (see Table 3) were 17.3 minutes and 29.8 minutes (flow rate of 10 mL/min), respectively. The S(+) form of MA5 was obtained with an optical purity of 100% by column separation. Chromatograms are shown in FIG. 17.

### 1-4 Determination of Absolute Configuration of S(+) Form of MA5

Measurement of the optical rotations of MA5 in the pre-elution fraction and MA5 in the post-elution fraction preparatively separated was carried out by using polarimeter P2200 (manufactured by JASCO Corporation).

The specific rotation of MA5 in the pre-elution fraction was [α]_{D}+130.3 (c=1.0, chloroform solution, 25° C.), and the specific rotation of MA5 in the post-elution fraction was [α]_{D}−128.6 (c=1.0, chloroform solution, 25° C.).

X-ray crystallography was carried out by using AXS “SMART APEX II” (manufactured by Bruker Corporation). As a crystal used for measurement, a crystal of a benzylamine salt of MA5 in each elution fraction was created because it is suitable for single crystal creation (see Table 4 and 5). MA5 in the pre-elution fraction or MA5 in the post-elution fraction and benzylamine were mixed at a molar ratio of 1:1 to prepare an ethyl acetate solution, a crude crystal was obtained from the ethyl acetate solution, and then a benzylamine salt of MA5 in each elution fraction was obtained by recrystallization from a water-methanol solution. The crystal structure was determined by an anomalous scattering effect, and the absolute configuration of the benzylamine salt of MA5 in the pre-elution fraction was S, whereas the absolute configuration of the benzylamine salt of MA5 in the post-elution fraction was R.

TABLE 1
Crystal data on S(+)-MA5 benzylamine salt:

C [symbol] H [symbol] F₂N₂O₃, (M = 436.44 g/mol), monoclinic, space group P2₁ (no. 4),
a = 11.0175(4) Å, b = 6.6046(2) Å, c = 15.0931(5) Å, β = 106.9980(10)°, V =
1050.29(6) Å³, Z = 2, T = 293.15 K, μ(CuKα) = 0.860 mm [symbol] , Dcalc = 1.380 g/cm³,
crystal size 0.2 × 0.15 × 0.15 mm³, F(000) = 456, 35841 reflections measured
(8.826° ≤ 2Θ ≤ 149.292°), 4210 unique (Rᵢₙₜ = 0.0369, R [symbol] = 0.0255) which were
used in all calculations. The final R₁ was 0.0263 (I > 2σ (I)) and wR₂ was 0.0699
(all data). The goodness of fit on F² was 1.082. Flack parameter = 0.00(2). [symbol]
indicates data missing or illegible when filed

TABLE 2
Crystal data on R (−)-MA5 benzylamine salt:

C₂₅H₂₃F₃N₃O₃, (M = 436.44 g/mol), monoclinic, space group P2₁ (no. 4),
a = 11.0185(3) Å, b = 6.6030(2) Å, c = 15.0857(5) Å, β = 106.9090(10)°, V =
1050.11(6) Å³, Z = 2, T = 100(2) K, μ(CuKα) = 0.860 mm⁻¹, Dcalc = 1.380 g/cm³,
crystal size 0.2 × 0.1 × 0.1 mm³, F(000) = 456, 21786 reflections measured
(8.832° ≤ 2Θ ≤ 149.224°), 4116 unique (Rᵢₙₜ = 0.0308, R [symbol] = 0.0254) which were
used in all calculations. The final R₁ was 0.0263 (I > 2σ (I)) and wR₂ was 0.0677
(all data). The goodness of fit on F² was 1.066. Flack parameter = 0.036(18). [symbol]
indicates data missing or illegible when filed

From the above results, it was determined that MA5 in the pre-elution fraction was the S(+) form and that MA5 in the post-elution fraction was the R(−) form.

TABLE 3

| Compound name | Structural formula |
|---|---|
| Racemic form of MA5 | 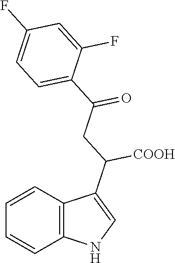 [SMILES: O=C(CC(C(=O)O)c1c[nH]c2ccccc12)c1ccc(F)cc1F] |
| S(+) form of MA5 | 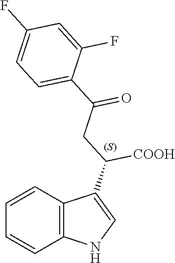 |
| R(−) form of MA5 | 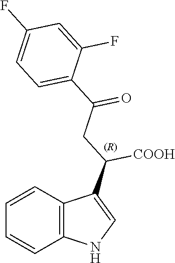 |

TABLE 4

| Compound name | Structural formula | |
|---|---|---|
| Benzylamine salt of S(+) form of MA5 | 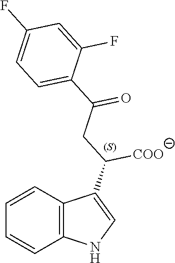 | 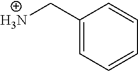 |
| Benzylamine salt of R(−) form of MA5 | 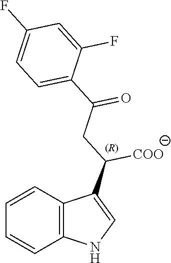 | 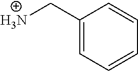 |

TABLE 5

| Compound name | Structural formula | |
|---|---|---|
| ORTEP diagram of benzylamine salt of S(+) form of MA5 | 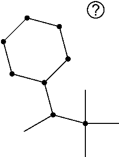 | 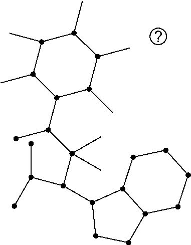 |
| ORTEP diagram of benzylamine salt of R(−) form of MA5 | 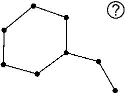 | 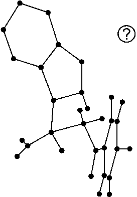 |

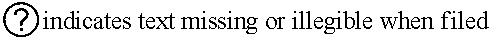

### 2. Evaluation of Amount of NAD⁺ in Animal Cells

In order to investigate the influence of the S(+) form of MA5 on the amount of NAD⁺ in animal cells, analysis was carried out according to the method described in the section “2-1” below.

### 2-1 Method

An INS-1E cell line (see the literature “Drug Metab. Pharmacokinet. 25 (3): 274-282 (2010)”) was seeded in a lysine-coated 96-well plate at 2×10⁴ cells per well, and cultured for 48 hours in the presence of RPMI-1640 Medium, then the Medium was removed, RPMI-1640 Medium containing 0.1% DMSO, which is a solvent for the compounds, or RPMI-1640 Medium containing various concentrations (3 pM, 10 pM, 30 pM, 100 pM, 300 pM, 1 nM, or 3 nM) of three compounds (racemic form of MA5, S(+) form of MA5, or R(−) form of MA5) were added in an amount of 100 μL each, and the cells were cultured for 6 hours. After that, the amount of NAD⁺ in the cells was measured by using NAD/NADH-Glo Assay (G9071, manufactured by Promega Corporation).

### 2-2 Results

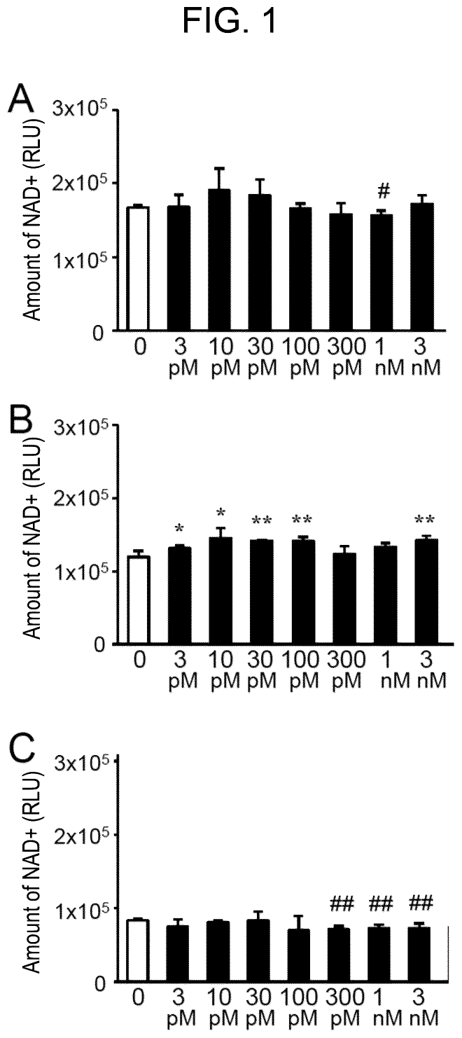

Even when the INS-1E cell line was cultured in the presence of the racemic form of MA5, the amount of NAD⁺ in the cells remained almost unchanged as compared with when the INS-1E cell line was cultured in the absence of the racemic form of MA5 (see FIG. 1A). On the other hand, when the INS-1E cell line was cultured in the presence of the R(−) form of MA5, the amount of NAD⁺ in the cells decreased in a culture time-dependent manner as compared with when the INS-1E cell line was cultured in the absence of the R(−) form of MA5 (see FIG. 1C). In addition, when the INS-1E cell line was cultured in the presence of the S(+) form of MA5, the amount of NAD⁺ in the cells increased in a culture time-dependent manner as compared with when the INS-1E cell line was cultured in the absence of the S(+) form of MA5 (see FIG. 1B).

These results indicate that the R(−) form of MA5 has the action of decreasing the amount of NAD⁺ in animal cells, whereas on the contrary, the S(+) form of MA5 has the action of increasing the amount of NAD⁺ in animal cells (that is, the action of promoting generation of NAD⁺ in animal cells).

### 3. In Vivo Administration Test 1

When the S(+) form of MA5 was administered in vivo, whether or not chiral conversion occurred and changes in the concentration of the S(+) form of MA5 in plasma were analyzed according to the method described in the section “3-1” below.

### 3-1 Method

Each enantiomer (S(+) form or R(−) form) of MA5 was dissolved in a mixed solution of physiological saline and 0.1 N sodium hydroxide (9:1), and filtered and sterilized by using a 0.22 μm filter to prepare a solution containing 2 mL/kg of each enantiomer of MA5. Next, the solution containing each enantiomer of MA5 was intravenously administered to each of three 4- to 5-year-old male cynomolgus monkeys at a single dose of 1 mg/kg. As a control, a solution containing no enantiomer of MA5 was also administered in the same manner. Blood was collected 5 minutes, 15 minutes, 30 minutes, 1 hour, 2 hours, 4 hours, 6 hours, and 24 hours after administration to collect plasma, the concentration of each enantiomer of MA5 in the plasma was measured by LC-MS/MS (liquid chromatography/tandem mass spectrometry), and the area under the blood concentration-time curve (AUC) (μg·hr/mL) was calculated. The analysis conditions for LC-MS/MS are as follows.

As an ionization unit, an apparatus in which NANOSPACESI-2 (manufactured by OSAKA SODA CO., LTD.) as a high-performance liquid chromatograph was connected to a triple quadrupole mass spectrometer (TSQ Quantum Ultra, manufactured by Thermo Fisher Scientific Inc.) equipped with an ESI probe was used. The S(+) form of MA5 (S-(+)-MA5) and the R(−) form of MA5 (R-(−)-MA5), which are substances to be analyzed, were subjected to quantitative analysis by the internal standard method using their respective stable isotopes S-(+)-MA5-d₆ and R-(−)-MA5-d₆. CHIRALPAK AD-3R (150×2.1 mm i.d., 3.0 μm particle size, manufactured by Daicel Corporation) was used as the analytical column, and CAPCELL C8UG120 (10×2.0 mm i.d., 5.0 μm particle size, manufactured by OSAKA SODA CO., LTD.) was used as the guard column. The column oven temperature was 35° C. Formic acid/water (0.1/100 [v/v]) (solution A)/acetonitrile (solution B) was used as the mobile phase, and the flow rate was 200 μL/min. The measurement was carried out under an isocratic condition in which a single composition of solution A/solution B (57/43 [v/v]) was used for elution. Solution A/solution B (57/43 [v/v]) was used until the analysis time reached 9.5 minutes, and solution A/solution B (10/90 [v/v]) was used after the analysis time reached 9.7 minutes. In addition, a system for washing the column with solution A/solution B (10/90 [v/v]) was incorporated while the analysis time elapsed was 9.7 minutes to 11.7 minutes, and the column was equilibrated with solution A/solution B (57/43 [v/v]) while the analysis time elapsed was 11.8 minutes to 15 minutes. The retention time was 6.95 minutes for S-(+)-MA5, 6.80 minutes for S-(+)-MA5-d₆, 8.69 minutes for R-(−)-MA5, and 8.56 minutes for R-(−)-MA5-d₆.

The MS conditions were detected in negative ion mode, a spray voltage set to 2.5 kV, a vaporizer temperature set to 250° C., and an ion transfer tube temperature set to 250° C. The precursor ion>product ion SRM channel was set to “m/z 328.0>116.2” for MA5 and “m/z 334.0>121.1” for MA5-d₆. The collision energy for generating a product ion was set to 12 eV for MA5 and 17 eV for MA5-d₆. Waveform analysis and quantitative calculation by the internal standard method were carried out by using Xcalibur (manufactured by Thermo Fisher Scientific Inc.).

### 3-2 Results

From immediately after administration of the S(+) form of MA5 (5 minutes after administration) to 24 hours after administration when the S(+) form of MA5 disappeared in blood, the R(−) form of MA5 was not detected, and only the S(+) form of MA5 was detected. This result indicates that the S(+) form of MA5 does not cause chiral conversion in vivo.

In addition, the AUC value when the R(−) form of MA5 was administered was 12 (μg·hr/mL), whereas the AUC value when the S(+) form of MA5 was administered was 30 (μg·hr/mL) (see Table 6). This result indicates that the S(+) form of MA5 is 2.5 times more likely to be retained in blood than the R(−) form of MA5, suggesting that the dosage of the S(+) form of MA5 can be made lower than that of the R(−) form of MA5.

TABLE 6

| | t_{1/2} | ke | AUC |
|---|---|---|---|
| | (hr) | (hr⁻¹) | (μg/mL · hr) |
| S(+) form of MA5 | 2.4 | 0.29 | 30 |
| R(−) form of MA5 | 1.1 | 0.63 | 12 |

### 4. Metabolism Test Using Human Liver Microsomes

The S(+) form of MA5 was more likely to be retained in blood than the R(−) form of MA5 in vivo, and thus whether or not there was a difference in metabolic rate between the enantiomers of MA5 was analyzed according to the method described in the section “4-1” below.

### 4-1 Method

The S(+) form or the R(−) form of MA5 was dissolved in acetonitrile (for LC-MS, manufactured by Kanto Chemical Co., Inc.) and each adjusted to 10 mM. 855 mg of sucrose (manufactured by FUJIFILM Wako Pure Chemical Corporation) was dissolved in 10 mL of water to prepare a 250 mM sucrose solution, and then by using this solution, a liquid containing 20 mg/mL human liver microsomes (8-donor-pool, manufactured by Sekisui XenoTech, LLC) was diluted 4-fold to prepare a 5 mg/mL human liver microsome suspension. In a 1 mL tube, 5 μL of three test solutions (sucrose solution containing 10 mM S(+) form of MA5, sucrose solution containing 10 mM R(−) form of MA5, or control Vehicle [sucrose solution]), 5 μL of NADPH Regenerating System Solution B (manufactured by Corning Incorporated), 100 μL of UGT Reaction Mix Solution B (manufactured by Corning Incorporated), and 275 μL of water were added to 50 μL of the human liver microsome suspension, and the resulting mixture was shaken for 30 seconds and preincubated at 37° C. for 5 minutes. After that, 25 μL of NADPH Regenerating System Solution A (manufactured by Corning Incorporated) and 40 μL of UGT Reaction Mix Solution A (manufactured by Corning Incorporated) were added, and the resulting mixture was shaken for 30 seconds and incubated at 37° C. for 1 hour. The reaction was stopped by adding 500 μL of an ice-cold acetonitrile solution containing 0.1% formic acid, and the resulting mixture was shaken for 30 seconds, sonicated for 10 minutes, and then centrifuged at 20,000×g at 4° C. for 10 minutes. 50 μL of the supernatant was collected in a fresh 1 mL tube, centrifugally concentrated at 2,000 rpm at room temperature (25 to 35° C.) for 30 minutes, and frozen at −80° C. This was dried with a freeze dryer for 30 minutes and redissolved in 25 μL of 20% methanol containing 0.1% formic acid (for LC-MS, manufactured by Kanto Chemical Co., Inc.), and then 2 μL thereof was subjected to LC/HRMS. The number of samples was n=3, and operations were carried out on ice as much as possible.

### LC/HRMS Measurement

Nanospace si-2 (manufactured by OSAKA SODA CO., LTD.) was used as an LC apparatus. Develosil C30-ug-3 (2.1×150 mm, 3.0 μm particle size, manufactured by Nomura Chemical Co., Ltd.) was used as a column, 10 mM NH₅CO₃/H₂O was used as the mobile phase solution A, methanol was used as the mobile phase solution B, and linear gradient elution was carried out at a flow rate of 200 μL/min (15 to 65% solution B (0.0 to 22.0 minutes), 65 to 90% solution B (22.0 to 24.4 minutes), 100% solution B (24.5 to 29.0 minutes), 15% solution B (30.0 to 40.0 minutes)). The column oven was set to 40° C., the sample injection volume was set to 2 μL, 10% methanol was selected as the wash solution, and 50% acetonitrile was selected as the wash port solution.

Q-Exactive (manufactured by Thermo Fisher Scientific Inc.) was used as an HRMS apparatus. The ESI conditions set were spray voltage: 3000 V, sheath gas pressure: 40 psi, auxiliary gas pressure: 10 psi, and capillary temperature/vaporizer temperature: 300° C. Full scan-ddMS² (topN) mode for both positive and negative ions was selected for detection. Various parameters are as follows: Full scan; Resolution 70,000, AGC target 3e6, Maximum IT 100 ms, Scan range 80 to 1,200, ddMS²; Resolution 17,500, AGC target 5e4, Maximum IT 50 ms, topN N=20, Isolation window 4.0 m/z, and NCE 20 (50% stepped NCE). Additional MS² data were measured in target MS² mode for both positive and negative ions. Various parameters are as follows: Resolution 35,000, AGC target 6e5, Maximum IT 200 ms, Isolation window 4.0 m/z, and NCE 20 (50% stepped NCE).

### Data Analysis

Data analysis was carried out by using Compound Discoverer (manufactured by Thermo Fisher Scientific Inc.) and Xcalibur (manufactured by Thermo Fisher Scientific Inc.). Metabolism w/FlSh Workflow was selected for peak extraction, and structural information of MA5 was registered in “Compound.” Min. Peak Intensity was set to 1/10,000 of the MA5 average peak intensity in each sample. [M+Na]⁺, [M+NH₄]⁺, [M−H₂O+H]⁺, and [M+Cl]⁻ were added to Ion Adducts of Predict Metabolites Node. In addition, tryptophan>kynurenine pathway [—C+O], AMP conjugation [+C₁₀H₁₂N₅O₆P], CoA conjugation [+C₂₁H₃₄N₇O₁₅P₃S] were added to Metabolic Pathways.

### 4-2 Results

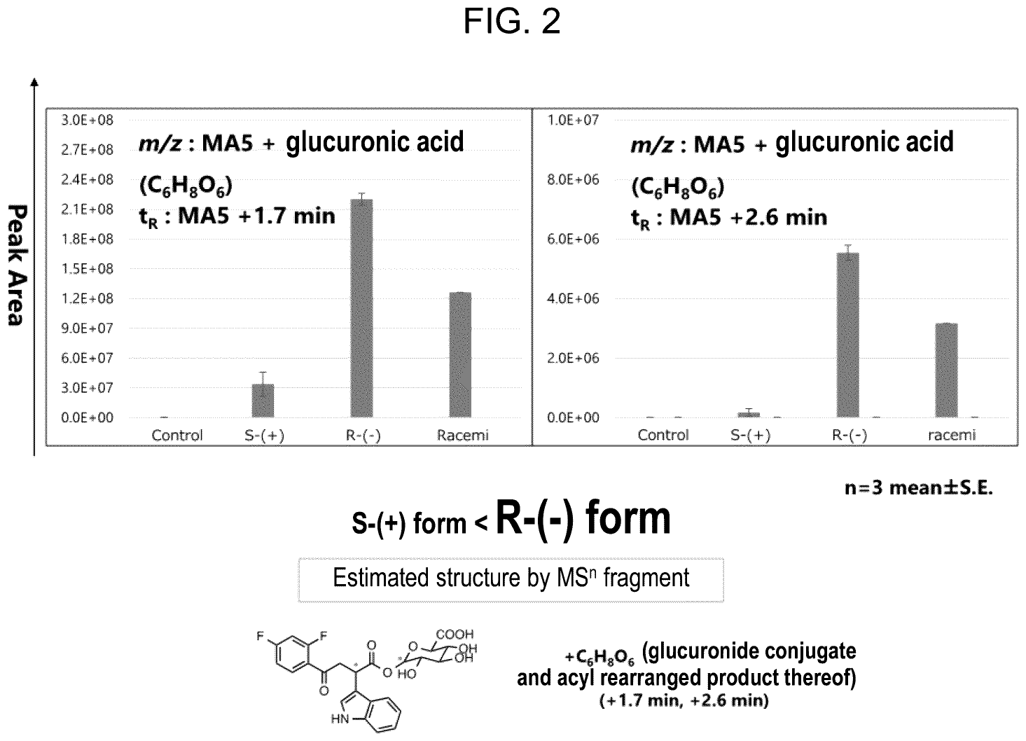

When human liver microsomes were incubated in the presence of the racemic form of MA5 or the R(−) form of MA5, glucuronide conjugates (+C6H8O6 [+1.7 min, +2.6 min]) were detected, whereas when human liver microsomes were incubated in the presence of the S(+) form of MA5, such glucuronide conjugates were detected only at a level that was little or no different from the control (see FIG. 2).

This result indicates that the R(−) form of MA5 is no less than 10 times more likely to be glucuronidated than the S(+) form of MA5, supporting the results of the section “4. Metabolism test using human liver microsomes” above, that is, the result that the S(+) form of MA5 was more likely to be retained in blood than the R(−) form of MA5 in vivo.

Furthermore, as a result of carrying out a 4-week repeated dose toxicity test in rats and a 4-week repeated dose toxicity test in monkeys from GLP-compliant non-clinical studies, a finding of toxicity to the urothelium was observed in the high-dose administration group. In order to clarify the cause thereof, urine metabolites were evaluated, and the urine concentrations of glucuronide conjugates of the racemic form of MA5 for monkeys and the urine concentrations of sulfate conjugates of the racemic form of MA5 for rats correlated with the development of toxicity, and it was presumed that these conjugates were responsible for toxicity.

It has been revealed that there are species differences between monkeys and rats in liver metabolic enzymes and renal transporters, and that hepatic metabolism in humans is similar to that in monkeys, and thus it is presumed that a glucuronide conjugate is also responsible for a side effect in humans. From the metabolism test using human liver microsomes, the S(+) form of MA5 produces almost no ( 1/10 or less) glucuronide conjugate as compared with the R(−) form, and thus it can be deemed that the S(+) form of MA5 is a safer compound with less side effects than the R(−) form of MA5.

### 5. Cytotoxicity Evaluation 1

In order to evaluate the toxicity of the S(+) form of MA5 to animal cells, analysis was carried out according to the method described in the section “5-1” below.

### 5-1 Method

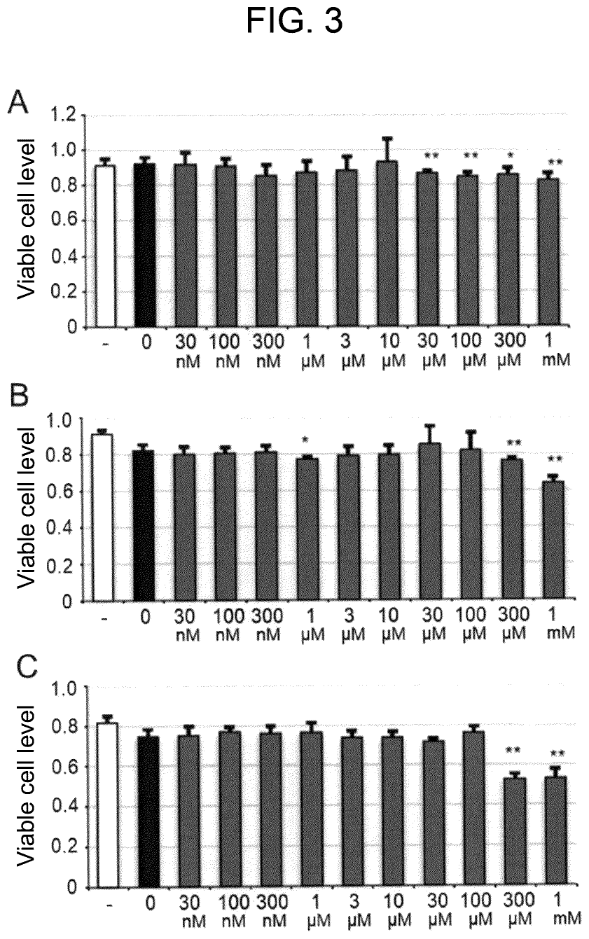

An HBlEpC cell line (manufactured by Cell Application) was seeded in a 96-well plate at 2×10³ cells per well, 0.1% DMSO, which is a solvent for the compounds, or various concentrations (30 NM, 100 nM, 300 nM, 1 μM, 3 μM, 10 μM, 30 μM, 100 μM, 300 μM, or 1 mM) of three compounds (racemic form of MA5, S(+) form of MA5, or R(−) form of MA5) were added to the HBlEpC culture medium, and the cells were cultured for 48 hours. As a control, an experiment in which nothing was added was also carried out (“-” in FIG. 3). The viable cell level (viable cell count level) after culture was measured by a WST-8 assay using Cell Count Reagent SF (manufactured by Nacalai Tesque, Inc.).

### 5-2 Results

When the HBlEpC cell line was cultured in the presence of an enantiomer (S(+) form or R(−) form) of MA5, the viability of the cells was higher than when the HBlEpC cell line was cultured in the presence of the racemic form of MA5, and in particular, the viability of the cells was higher with the S(+) form of MA5 than with the R(−) form of MA5 (see FIG. 3). This result indicates that the S(+) form of MA5 has lower cytotoxicity to animal cells than the racemic form of MA5 and the R(−) form of MA5.

### 6. Cytotoxicity Evaluation 2

In order to evaluate the toxicity of the S(+) form of MA5 to animal cells, analysis was carried out according to the method described in the section “6-1” below.

### 6-1 Method

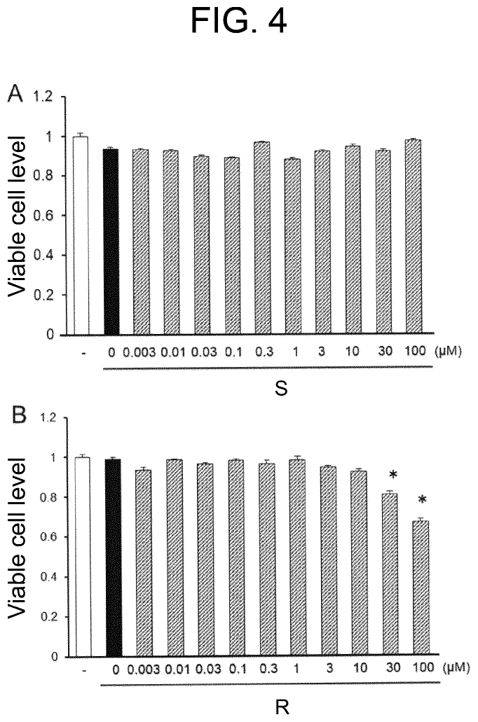

An MT-ND3 mutant cell line was seeded in a 96-well plate at 2×10³ cells per well and cultured for 2 days in the presence of DMEM Low Glucose culture medium, then 0.1% DMSO, which is a solvent for the compounds, or various concentrations (0.003 μM, 0.01 μM, 0.03 μM, 0.1 M, 0.3 μM, 1 μM, 3 μM, 10 μM, 30 μM, or 100 μM) of each enantiomer (S(+) form or R(−) form) of MA5 was added, the cells were further cultured for 5 days, and then the cell survival level was measured. Specifically, the cell survival level was measured by an MTT assay using Cell Counting Kit-8 (manufactured by Dojindo Laboratories). That is, 100 μL of Cell Count Reagent SF was added to each well, incubated for 2 hours, and stirred for 3 seconds with a microplate reader, and the absorbance at 450 nm (reference: 750 nm) was measured (see FIG. 4).

As a control, an experiment in which nothing was added was also carried out (“-” in FIG. 4).

### 6-2 Results

Even when the MT-ND3 mutant cell line was cultured in the presence of the S(+) form of MA5 at high concentrations (30 μM and 100 μM), no decrease in viable cell level was observed (see FIG. 4A). On the other hand, when the MT-ND3 mutant cell line was cultured in the presence of the R(−) form of MA5 at high concentrations (30 μM and 100 μM), a significant decrease in viable cell level was observed (see FIG. 4B). These results indicate that the S(+) form of MA5 has lower cytotoxicity to animal cells than the R(−) form of MA5, supporting the results of the section “5. Cytotoxicity evaluation 1” above.

### 7. Evaluation of Ability to Produce ATP in Animal Cells

It has been reported that the racemic form of MA5 has the action of enhancing the ability to produce ATP in animal cells (see, for example, International Publication No. WO 2014/080640). Therefore, in order to check whether or not there is a difference in the action of enhancing the ability to produce ATP between the S(+) form of MA5 and the R(−) form of MA5, analysis was carried out according to the method described in section “7-1” below.

### 7-1 Method

An MT-ND3 mutant cell line was seeded in a 96-well plate at 2×10³ cells per well and cultured for 24 hours in the presence of DMEM Low Glucose culture medium, then 0.1% DMSO, which is a solvent for the compounds, or various concentrations (0.1 μM or 1 μM) of three compounds (racemic form of MA5, S(+) form of MA5, or R(−) form of MA5) were added, and the cells were cultured for 6 hours, and then the concentration of ATP produced in the culture medium was measured with GloMa 96 Microplate Luminometer (Promega Corporation) by using a “cellular” ATP measurement reagent (manufactured by TOYO B-Net Co., Ltd.).

### 7-2 Results

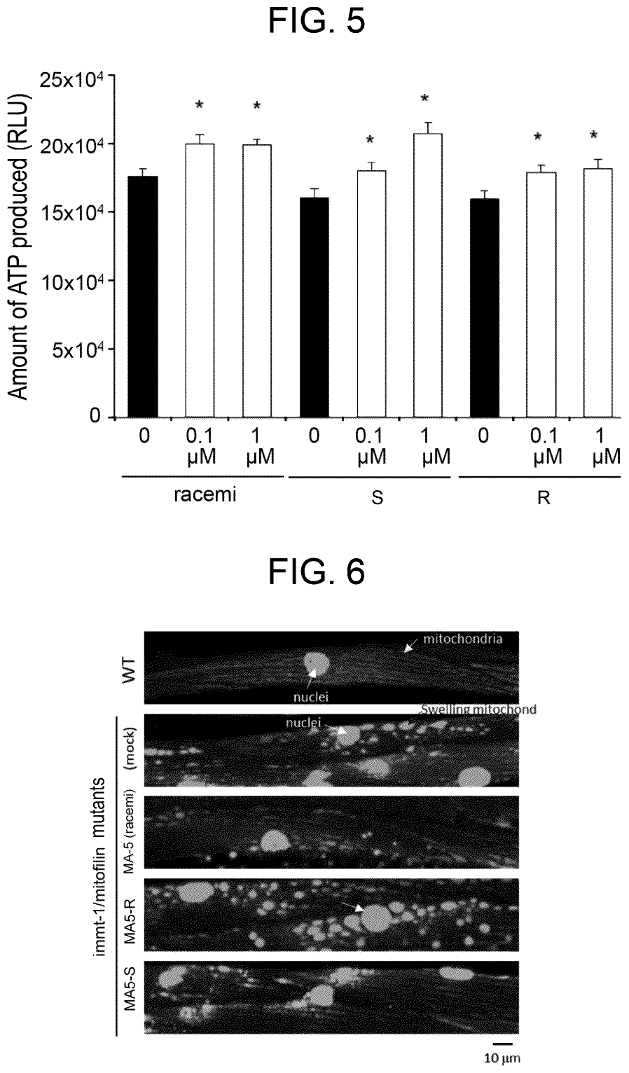

No difference was observed in the level of ATP production in animal cells between the S(+) form of MA5 and the R(−) form of MA5 (see FIG. 5).

### 8. Evaluation of Lifespan

In order to confirm that the S(+) form of MA5 has the effect of extending the lifespan of an organism, analysis is carried out, for example, according to the methods described in the sections [Method 1] and [Method 2] below.

### [Method 1]

[1] Neurodegeneration model flies (obtained from the Bloomington Drosophila Stock Center) are crossed, and the hatched eggs are raised at 18° C.
[2] One to three days after emergence into adults, the adults are placed in an incubator at 29° C.
[3] Vials containing a 5% sucrose solution or a 5% sucrose solution containing various concentrations (100 nM or 1000 nM) of three compounds (racemic form of MA5, S(+) form of MA5, or R(−) form of MA5) are provided, and 20 male (♂) flies and female (♀) flies are transferred to each vial and allowed to ingest each solution (for 20 hours at 29° C., with protection from light).
[4] The flies are transferred to another vial containing regular food and allowed to ingest the regular food (for 1 day).
[5] From the next day onward, the above steps [3] and [4] are repeated.
[6] In the above steps [3] and [4], the dead flies are removed, the number thereof is recorded, and the lifespan of the dead flies is recorded. Once the vial is emptied (that is, there are no live flies in the vial), the number of survivors is calculated by taking the sum of the recorded numbers of dead flies as the original number, and the average lifespan of the flies is calculated based on the lifespans of the dead individual flies and the total number of surviving flies. The reason for changing the temperature from 18° C. to 29° C. is that a temperature-sensitive gene expression system is used, and that no neurodegenerative proteins are expressed during the developmental process, and neurodegenerative proteins are expressed after emergence into adults (expression of neurodegenerative proteins in all neurons during the developmental process is often lethal).

Flies have been used as a model organism for a human disease (see the literature “Hum Mol Genet. 2019. PMID: 31227826”) or used as a model organism for human aging (see the literature “FEBS Open Bio. 2022. PMID: 34854258”). Because of this, if the lifespan extension effect of the S(+) form of MA5 is observed in flies, it can be deemed that the effect can also be applied to a human.

### [Method 2]

[1] 28-day-old α-klotho homo KO mice (male [♂], 4 weeks old, n=3, 2 repeats) are obtained from CLEA Japan, Inc. and acclimatized to the environment for 2 days.
[2] On day 30 after birth, three compounds (racemic form of MA5, S(+) form of MA5, or R(−) form of MA5) suspended in water are orally administered to the above KO mice at a dose of 10 mg/kg body weight/100 μL/day.
[3] The body weights of the KO mice are measured once a week and recorded until the death thereof is confirmed. As a control, the body weights of KO mice to which the above three compounds have not been orally administered are similarly measured until the death thereof is confirmed.

### 9. Evaluation of Mitochondrial Function

It has been reported that in immt-1-deficient mutant C. elegans (hereinafter referred to as “immt-1/mitofilin mutant”), which is a C. elegans ortholog of the mitochondrial inner membrane protein (Mitofilin), mitochondria in muscle cells swell (see the literature “Journal of Cellular Physiology. 224 (2010) 748-756” and the literature “Molecular Biology of the Cell. 22 (2011) 831-841.”). Therefore, when three compounds (racemic form of MA5, S(+) form of MA5, or R(−) form of MA5) were administered to immt-1/mitofilin mutants, whether or not mitochondrial swelling was alleviated was analyzed.

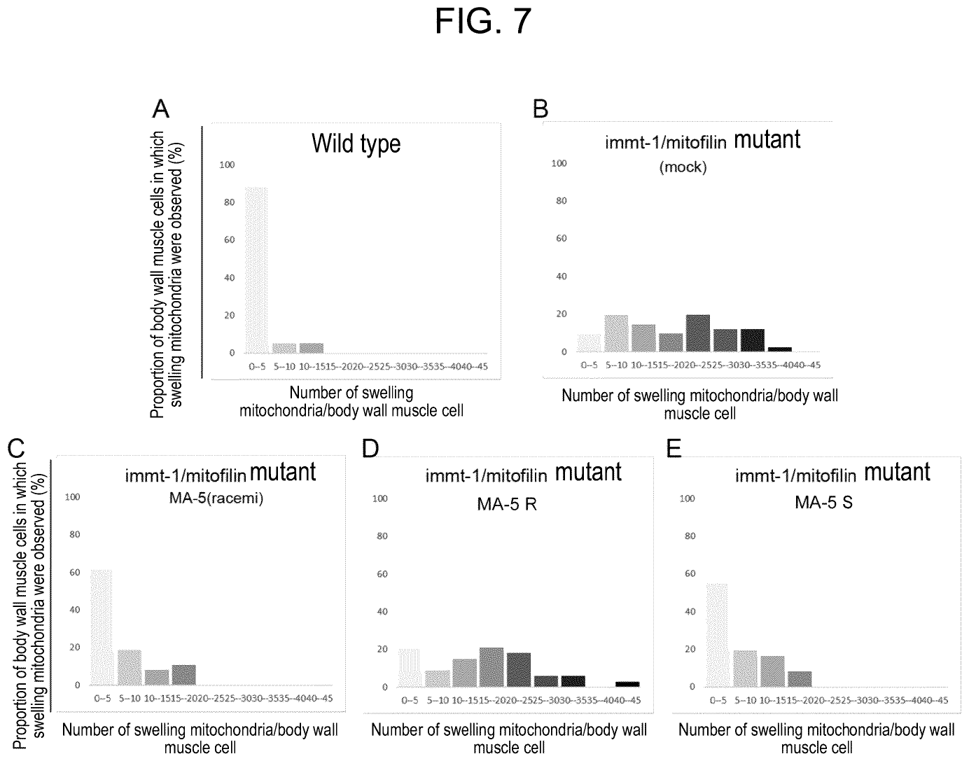

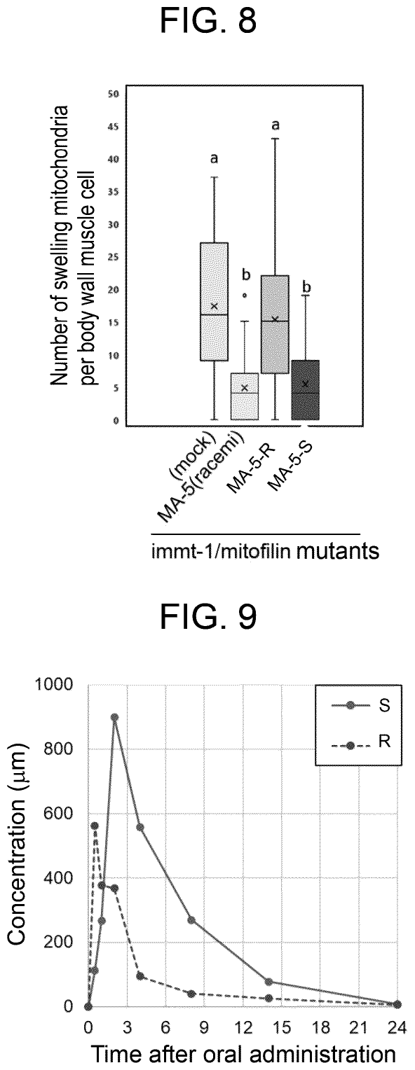

Specifically, immt-1/mitofilin mutants (NBRP [National Bio Resource Project] C. elegans [Tokyo Women's Medical University]) of ccIs4251 (Pmyo-3::GFP::LacZ::NLS, Pmyo-3::mitochondrial-GFP+dpy-20(+), the literature “Cell 139 (2009) 623-633”) genetically engineered to be able to visualize mitochondrial DNA and genomic DNA with GFP were raised for 5 days from the L4 larval stage (that is, until these became 4-day-old adults) on a culture plate (E. coli OP-50 NGM agar medium) that was sprayed with the control DMSO (solvent), or the above culture plate that was sprayed with three compounds (racemic form of MA5, S(+) form of MA5, or R(−) form of MA5) such that the final concentration was 10 μM, body wall muscle cells were observed in the 4-day-old adults (see FIG. 6), and the number of abnormal mitochondria (swelling mitochondria) and the proportion of body wall muscle cells in which abnormal mitochondria were observed were measured (see FIGS. 7 and 8). As a comparative control, ccIs4251 wild-type C. elegans (obtained from CGC [Caenorhabditis Genetics Center]) was raised on a culture plate and analyzed in the same manner.

As a result, when the immt-1/mitofilin mutants were raised in the presence of the S(+) form of MA5, the proportion of body wall muscle cells in which 5 or more swelling mitochondria per body wall muscle cell were observed greatly decreased (see FIG. 7), and the number of swelling mitochondria per body wall muscle cell also greatly decreased (see FIG. 8), as compared with when the mutants were raised in the presence of the control DMSO and when the mutants were raised in the presence of the R(−) form of MA5. In addition, when the immt-1/mitofilin mutants were raised in the presence of the R(−) form of MA5, the proportion of body wall muscle cells in which 5 or more swelling mitochondria per body wall muscle cell were observed and the number of swelling mitochondria per body wall muscle cell remained almost unchanged or only slightly decreased, as compared with when the mutants were raised in the presence of the control DMSO (see FIGS. 7 and 8).

These results indicate that the R(−) form of MA5 has almost no action of alleviating mitochondrial swelling, whereas the S(+) form of MA5 has the action of alleviating mitochondrial swelling. That is, the results indicate that the S(+) form of MA5 has the action of improving a mitochondrial function in C. elegans. In addition, C. elegans has been used as a model organism for analyzing a human mitochondrial function (see the literature “Nature. 2018. PMID: 30356218”), and thus it can be deemed that the S(+) form of MA5 has been shown to be useful as a mitochondrial function activating agent and a therapeutic agent or a preventive agent for a mitochondrial disease in a human.

### 10. In Vivo Administration Test 2

In “3. In vivo administration test 1” above, it was confirmed that the intravenously administered S(+) form of MA5 is more likely to be retained in blood than the intravenously administered R(−) form of MA5, and whether or not the same results were obtained even when these were orally administered was checked. Specifically, according to the method described in “3-1 Method” above, a solution containing each enantiomer (S(+) form or R(−) form) of MA5 was orally administered to male cynomolgus monkeys at a single dose of 100 mg/kg, the concentration of each enantiomer of MA5 and the concentration of a reduced metabolite of each enantiomer of MA5 (the compound represented by formula (B) [that is, Red-MA5]) in plasma were measured.

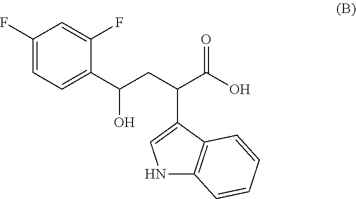 [SMILES: O=C(O)C(CC(O)c1ccc(F)cc1F)c1c[nH]c2ccccc12]

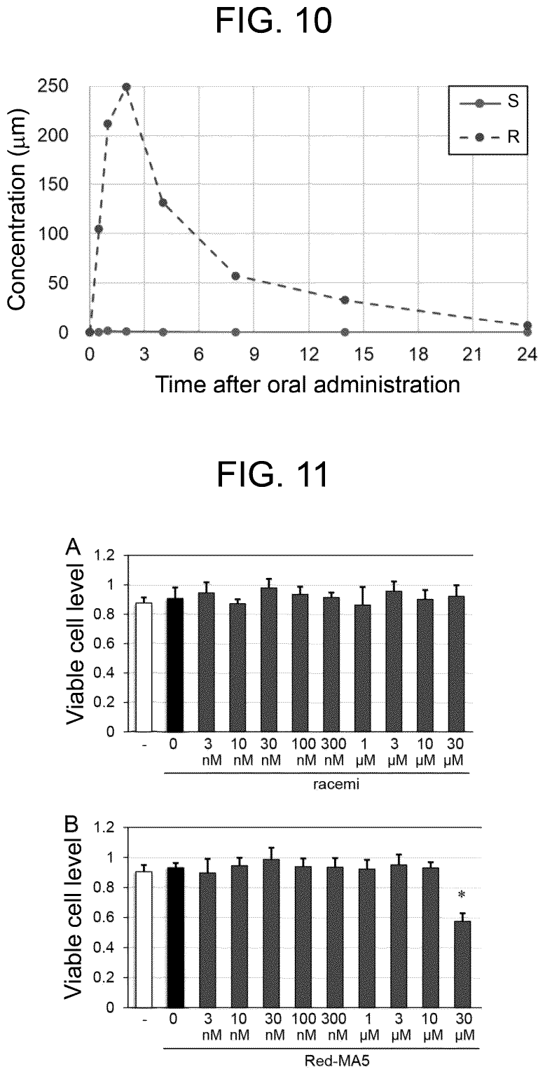

As a result, after oral administration, the concentration of the S(+) form of MA5 in plasma was higher than that of the R(−) form of MA5 in plasma (see FIG. 9). In addition, the reduced metabolite (the compound represented by formula (B), that is, Red-MA5) in plasma was detected when the R(−) form of MA5 was orally administered, whereas almost no reduced metabolite was detected when the S(+) form of MA5 was orally administered (see FIG. 10).

These results indicate that even when the S(+) form of MA5 is orally administered, the S(+) form is more likely to be retained in blood than is the R(−) form when the R(−) form of MA5 is orally administered, and the S(+) form of MA5 is less likely to be metabolized than the R(−) form of MA5, and thus the S(+) form of MA5 can exert the effect thereof in vivo more sustainably than the R(−) form of MA5.

### 11. Cytotoxicity Evaluation 3

In order to evaluate the toxicity of Red-MA5 to animal cells, analysis was carried out according to the method described in the section “11-1” below.

### 11-1 Method

A KCMC10 cell line was seeded in a 96-well plate at 3×10³ cells per well, and cultured for 24 hours in the presence of 0.1% DMSO, which is a solvent for the compounds, or various concentrations (3 nM, 10 NM, 30 nM, 100 nM, 300 nM, 1 μM, 3 μM, 10 μM, or 30 μM) of two compounds (racemic form of MA5 or Red-MA5), and then the cell survival level was measured. Specifically, the cell survival level was measured by an MTT assay using Cell Counting Kit-8 (manufactured by Dojindo Laboratories). That is, 100 μL of Cell Count Reagent SF was added to each well, incubated for 2 hours, and stirred for 3 seconds with a microplate reader, and the absorbance at 450 nm (reference: 750 nm) was measured (see FIG. 11). As a control, an experiment in which nothing was added was also carried out (“-” in FIG. 11).

### 11-2 Results

Even when the KCMC10 cell line was cultured in the presence of the 30 μM racemic form of MA5, no decrease in viable cell level was observed (see FIG. 11A), whereas when the KCMC10 cell line was cultured in the presence of M Red-MA5, a significant decrease in viable cell level was observed (see FIG. 11B). This result indicates that MA5 has lower cytotoxicity than Red-MA5. That is, the result, combined with the results of “10. In vivo administration test 2” above, indicates that the S(+) form of MA5 administered in vivo has lower cytotoxicity than the R(−) form of MA5 administered in vivo.

### 12. Evaluation of SIRT Expression Level in Animal Cells

It is known that there is a positive correlation between the amount of NAD⁺ and the SIRT expression level in cells (see the literature “Nat Rev Nephrol. 2017. PMID: 28163307”). In addition, it has been reported that the increased expression of an SIRT in inner ear cells is associated with improvement in hearing loss (see the literature “Front Cell Dev Biol. 2021. PMID: 34869361,” the literature “Exp Cell Res. 2022 Jul. 11:113280.”, and the literature “Hindawi Neural Plasticity Volume 2021, Article ID 5520794”). Therefore, in order to investigate whether or not when the S(+) form of MA5 increased the amount of NAD⁺ in cells, the expression level of an SIRT in inner ear cells was increased, and this was useful for improving hearing loss, analysis was carried out according to the method described in the section “12-1” below.

### 12-1 Method

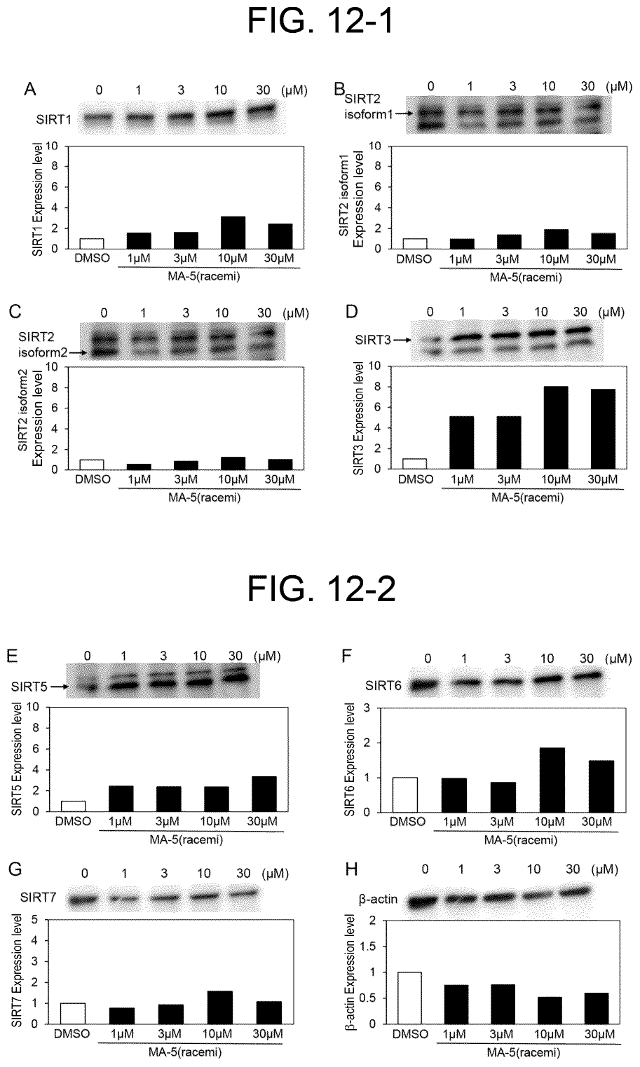

A mouse inner ear cell line (HEI-OC1 cell line) was seeded in a dish containing 10 mL of a culture medium (low glucose DMEM medium [glucose concentration of 1.0 g/dL] supplemented with 10% by weight of FBS) at 1×10⁶ cells/dish and cultured. One day after, 0.1% DMSO, which is a solvent for the compounds, or various concentrations (1 μM, 3 μM, 10 μM, or 30 μM) of three compounds (racemic form of MA5, S(+) form of MA5, or R(−) form of MA5) were added, and the cells were cultured for 24 hours. After the culture was completed, the cells were collected, the cells were disrupted by a conventional method, and the expression levels of various SIRTs (SIRT1, SIRT2, SIRT3, SIRT5, SIRT6, and SIRT7) in the cells were analyzed by Western blotting (see FIGS. 12 to 14 [FIG. 12-1 to FIG. 14-2]).

### 12-2 Results

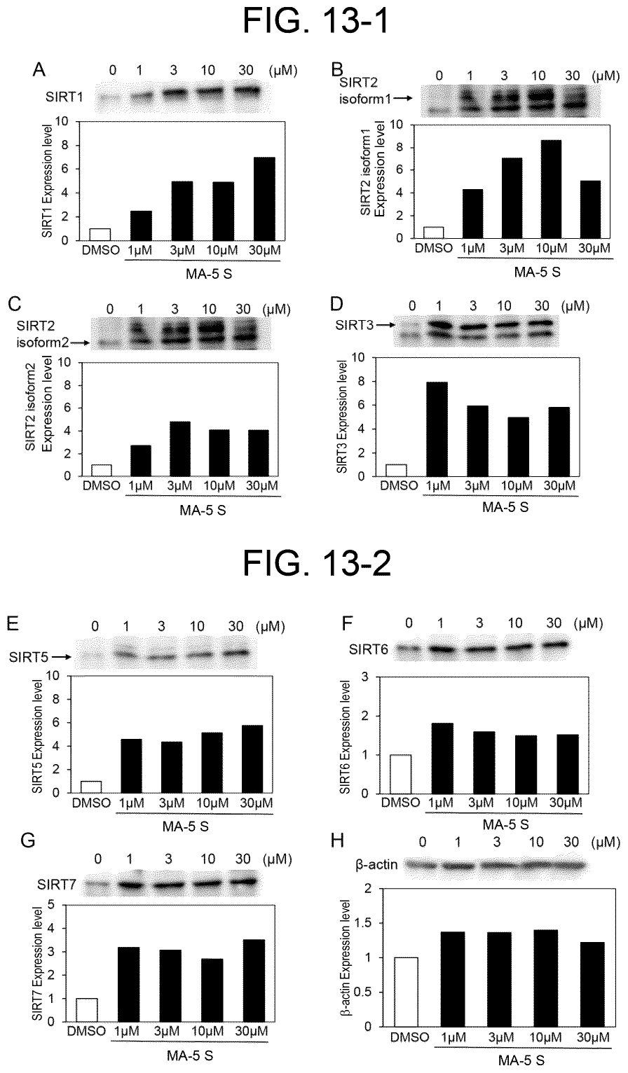

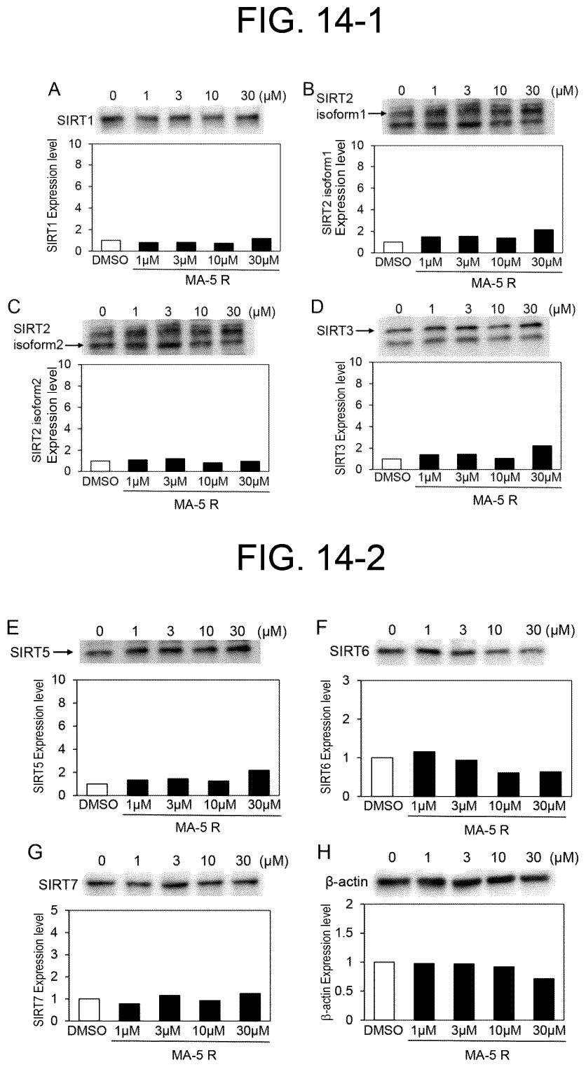

Even when the HEI-OC1 cell line was cultured in the presence of the R(−) form of MA5, the expression levels of the various SIRTs (SIRT1, SIRT2, SIRT3, SIRT5, SIRT6, and SIRT7) in the cells remained almost unchanged as compared with when the HEI-OC1 cell line was cultured in the absence of the R(−) form of MA5 (see FIG. 14 [FIG. 14-1 and FIG. 14-2]), whereas when the HEI-OC1 cell line was cultured in the presence of the S(+) form of MA5, the expression levels of the various SIRTs in the cells increased as compared with when the HEI-OC1 cell line was cultured in the absence of the S(+) form of MA5 (see FIG. 13 [FIG. 13-1 and FIG. 13-2]).

These results, combined with the results of “2. Evaluation of amount of NAD⁺ in animal cells” above, indicate that the S(+) form of MA5 increased the amount of NAD⁺ in the cells, and as a result, the expression levels of the SIRTs in the cells increased, and this is useful for improving hearing loss.

### 13. Evaluation of Motor Function in Animal

In order to confirm that the S(+) form of MA5 has the effect of improving motor dysfunction in an animal, for example, the mice described in the sections [Aging mice] and [Young mice] below are used to carry out analysis according to the methods described in the sections [Method 1] to [Method 3] below.

### [Aging Mice]

Water containing three compounds (racemic form of MA5, S(+) form of MA5, or R(−) form of MA5) is orally administered to aging mice (92 weeks) (5 mice) at a dose of 50 mg/kg body weight/day each for 9 weeks. In addition, as a control, water is orally administered to aging mice (6 mice) for 9 weeks.

### [Young Mice]

Water containing three compounds (racemic form of MA5, S(+) form of MA5, or R(−) form of MA5) is orally administered to young mice (4 months) (6 mice) at a dose of 10 mg/kg body weight/day (low dose) or 50 mg/kg body weight (high dose) each for 9 weeks. In addition, as a control, water is orally administered to young mice (6 mice) for 9 weeks.

### [Method 1: Treadmill Test]

[1] A treadmill apparatus for mice (MK-690, manufactured by Muromachi Kikai Co., Ltd.; hereinafter the same applies) is used to provide training for a treadmill test. Aging mice undergo a total of 3 days of training: day 1; twice×at a speed of 8 m/min for 10 minutes, day 2; twice×at a speed of 10 m/min for 10 minutes, and day 3; twice×at a speed of 12 m/min for 10 minutes. On the other hand, young mice undergo a total of 5 days of training: day 1; twice×at a speed of 8 m/min for 10 minutes, day 2; twice×at a speed of 9 m/min for 10 minutes, day 3; twice×at a speed of 10 m/min for 10 minutes; day 4; twice×at a speed of 11 m/min for 10 minutes, and day 5; twice×at a speed of 12 m/min for 10 minutes.
[2] The treadmill apparatus for mice is used to carry out the actual treadmill test for up to 2 hours under the following conditions. The test is started with a speed of 5 m/min, and the speed is increased by 5 m/min every 3 minutes, finally to a speed of 28 m/min (that is, 5 m/min→10 m/min→15 m/min→20 m/min→25 m/min→28 m/min).
[3] When the mouse does not move forward even when the tail is stimulated with a brush and does not move on an energized rod for 10 seconds or more, it is determined that the mouse has exhausted itself. When the mouse runs for more than 2 hours, the test is discontinued, and the time is recorded as 2 hours.

### [Method 2: Inverse Grating Suspension Test]

A mouse inverse grating suspension test is carried out by using a wire mesh that has a diameter of 37 cm and that is 8 mm square. A 12 cm plastic wall is constructed around the wire mesh for the purpose of preventing a mouse from escaping. A mouse is placed on the wire mesh for 1 minute for acclimatization. The wire mesh is turned upside down with the mouse in the center of the wire mesh, and the time (minutes) until the mouse falls is measured. The wire mesh is held at a height of 60 cm from the ground. In order to prevent the mouse from being injured when the mouse falls, bedding for raising is laid on the ground. When the mouse falls in less than 30 seconds, it is determined that the fall is an accidental fall, and an additional measurement is carried out until the mouse falls in 30 seconds or more. The measurement is carried out twice on another day, and the average value thereof is used. In addition, the body weight of the mouse is measured, and the “Hanging Impulse Score” (hanging time [minutes]×body weight [g]) is calculated.

### [Method 3: Grip Strength]

Mouse grip strength is measured by using a mouse grip strength measuring apparatus (GPM-101, manufactured by Melquest Ltd.). The grip strength of the front legs is measured by using a grip for the front legs, and the grip strength of the four legs is measured by setting a wire mesh for the four legs. The measurement is started by measuring the grip strength of the front legs, which is measured in the lateral direction and the longitudinal direction. For the measurement in the lateral direction, the mouse is pulled in the horizontal direction with both front legs of the mouse holding the grip until the paws are released. For the measurement in the longitudinal direction, the mouse is pulled in the vertical direction with both front legs of the mouse holding the grip until the paws are released. The maximum value of 5 consecutive measurements is measured. For the measurement for the four legs, the tail is pulled horizontally with the four legs of the mouse placed on the wire mesh until the paws are released, and the maximum value of 5 consecutive runs is measured.

### 14. Nampt Binding Test

The binding of the S(+) form of MA5 and the R(−) form of MA5 to nicotinamide phosphoribosyltransferase (Nampt), which is a key enzyme of the mammalian NAD⁺ synthesis system, was measured by using Octet K2.

A 0.01% DDM PBS solution was prepared as a measurement buffer. 1 μL of biotin Nampt was added to 1000 μL of the measurement buffer. This was diluted 2-fold to obtain a 0.4 ng/μL ligand solution. A DMSO solution of a 100 mM MA5 S(+) form was diluted from 300 M to 1000 μM, a DMSO solution of a 100 mM MA5 R(−) form was diluted from 500 μM to 1200 μM, and the diluted solutions were each used as an analyte solution. 200 μL each of the measurement buffer, a 1 μM biotin solution, a ligand solution, a 100 μM biocytin solution, and the analyte solution were added to a 96-well plate, and the plate was set in Octet K2 to carry out the measurement. Data were analyzed by using the accompanying software. Kd values were represented graphically with Prism 9 by using the concentrations of the analyte solution and the numerical values of Req (nm) calculated by the analysis.

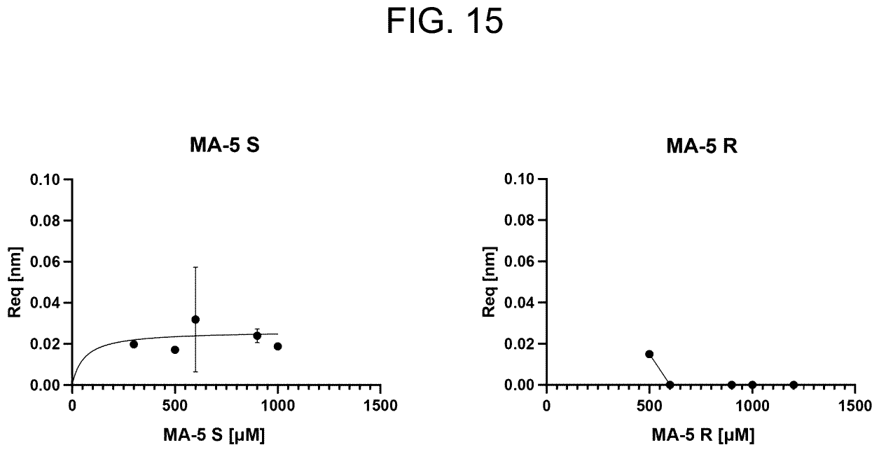

It was confirmed that the S(+) form of MA5 binds to Nampt, but the R(−) form does not bind to Nampt (FIG. 15). It is known that Nampt is an enzyme that controls the synthesis of NAD⁺, and thus the above result is consistent with the result that the S(+) form of MA5 increases the NAD⁺ level.

### 15. Effect of Suppressing DNA Damage Caused by Camptothecin

Rat fetal cardiomyoblast cells H9c2 were maintained and cultured in a DMEM medium (10% FBS, penicillin at 100 units/mL, streptomycin at 100 μg/mL). Camptothecin (CPT) at a final concentration of 10 μM, CPT at a final concentration of 10 μM, and MA5 (racemic form, S(+) form, or R(−) form) at a final concentration of 30 μM, or DMSO (control) were added. The cells were cultured for 24 hours, then the medium was removed, and γH2AX (phosphorylated H2AX), which is an indicator for DNA damage, was detected by immunostaining. The immunostaining was carried out by using a mouse anti-γH2AX antibody (Thermo Fisher) as a primary antibody and a CF568-labeled goat anti-mouse IgG antibody (Biotium Inc.) as a secondary antibody.

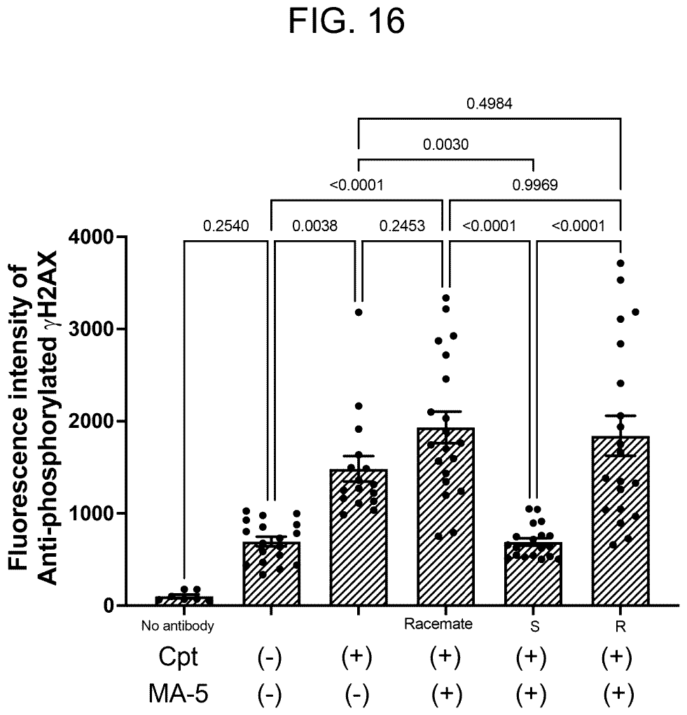

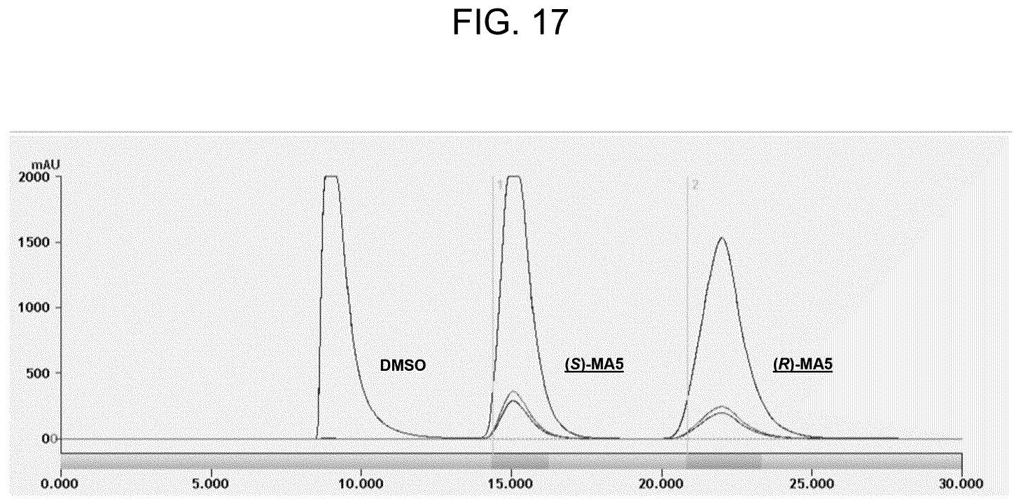

As a result of the immunostaining, a decrease in the fluorescence intensity of phosphorylated H2AX was observed only in the samples to which the S(+) form of MA5 was added (FIG. 16), confirming that the S(+) form of MA5 has the effect of suppressing DNA damage caused by CPT. The above effect was not observed for the R(−) form. It is known that an increase in NAD⁺ level has a DNA repair effect, and therefore, the above results suggest that the S(+) form of MA5 can suppress DNA damage caused by CPT through increasing the NAD⁺ level.

### 16. Physical Properties of S(+) Form of MA5

According to comparison of physical properties of the S(+) form of MA5 with those of the racemic form, the S(+) form of MA5 is easily soluble in acetone, acetonitrile, ethyl acetate, chloroform, dichloromethane, methanol, ethanol, and isopropanol. In addition, the S(+) form of MA5 is slightly soluble in a 50% acetonitrile aqueous solution, but sparingly soluble in a 50% methanol aqueous solution. On the other hand, the racemic form of MA5 is slightly soluble in a chloroform/methanol mixed solvent, a dichloromethane/methanol mixed solvent, and acetone, but sparingly soluble in ethyl acetate, chloroform, dichloromethane, and methanol.

In addition, the S(+) form of MA5 undergoes coloration and degradation under a light, acid, or alkali condition. Specifically, a dissolution solution of the S(+) form of MA5 in ethanol, isopropanol, acetone, acetonitrile, ethyl acetate, a 50% acetonitrile aqueous solution, or a 0.1% formic acid-containing acetonitrile solution is colorless and transparent, and does not become colored even after refrigerated for about a week. However, a dissolution solution obtained by dissolving the S(+) form of MA5 in methanol, dichloromethane, chloroform, a 50% methanol aqueous solution, or a 0.1% formic acid-containing methanol solution was colorless and transparent on the day of preparation, but colored pale yellow when refrigerated for about a week. Furthermore, a dissolution solution obtained by dissolving the S(+) form of MA5 in a mixed solvent of an alcohol (methanol, ethanol, or isopropanol) and chloroform was colored yellow the day after preparation.

A halogenated solvent generates hydrogen chloride as a slight amount of a degradation product, and thus, for example, chloroform includes ethanol as a stabilizer in an amount of 0.4 to 0.8%. It has been confirmed that a mixed solvent of an alcohol and chloroform promotes coloration and degradation, and it is considered that the presence of a slight amount of an acid and the presence of an alcohol may contribute to the degradation.

When a dissolution solution of the S(+) form of MA5 was spotted on silica gel TLC plate and developed after some time, a degradation product appeared, but when the dissolution solution was developed immediately, no degradation product was generated. A degradation product was not easily generated in an ethyl acetate dissolution solution, whereas a large amount of a degradation product was generated in an alcohol-chloroform dissolution solution.

TLC is under a slightly acidic condition, which may promote the degradation. Furthermore, a combination of an alcohol (short chain alcohol) and chloroform tends to promote degradation more.

### INDUSTRIAL APPLICABILITY

The present invention contributes to prevention or treatment of a symptom or a disease such as a symptom or a disease associated with aging, a metabolic disease, or a mitochondrial disease, and to the extension of the lifespan.

1. A compound of formula (A-1): 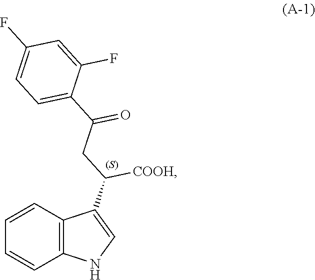
substantially free of an R(−) form, or a physiologically acceptable salt thereof.

2. The compound of claim 1, wherein an enantiomeric excess of an S(+) form is at least 97% or more, or a physiologically acceptable salt thereof.

3. A composition, comprising:
the compound of claim 1 or a physiologically acceptable salt thereof.

4. The composition of claim 3, wherein an enantiomeric excess of an S(+) form is at least 97% or more.

5. The composition of claim 3, which is stored with protection from light.

6. The composition of claim 3, which is stored near neutrality.

7. The composition of claim 3, which is stored at pH in a range of from 6.0 to 8.0.

8. The composition of claim 3, further comprising:
chloroform,
wherein a content of a short chain alcohol based on chloroform is less than 1%.

9. The composition of claim 3, which does not comprise a short chain alcohol and chloroform at the same time.

10. The composition of claim 3, further comprising:
a physiologically acceptable carrier.

11. The composition of claim 3, which is a pharmaceutical composition.

12. The composition of claim 3, which is a food or drink composition.

13. A method for treating, improving, or preventing a mitochondrial disease, the method comprising:
administering a compound of formula (A-1): 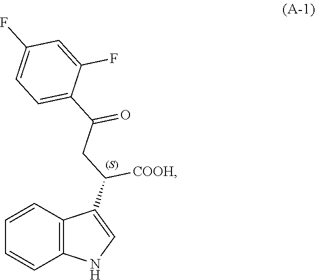
substantially free of an R(−) form, or a physiologically acceptable salt thereof to a subject in need thereof.

14. The method of claim 13, wherein an enantiomeric excess of an S(+) form is at least 97% or more.

15. The method of claim 13, wherein the administering increases an NAD⁺ level in the subject.

16. A method for treating, improving, or preventing a symptom or a disease associated with aging, the method comprising:
administering a compound of formula (A-1): 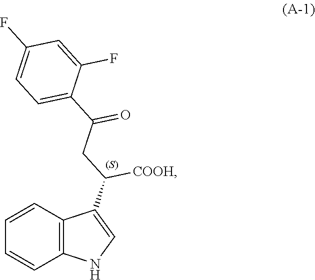
substantially free of an R(−) form, or a physiologically acceptable salt thereof to a subject in need thereof.

17. The method of claim 16, wherein an enantiomeric excess of an S(+) form is at least 97% or more.

18. The method of claim 16, wherein the symptom or the disease associated with aging is visual impairment, anemia, muscle weakness, thinning hair or hair loss, and/or shortened lifespan.

19. The method of claim 16, wherein the symptom or the disease associated with aging is hearing loss.

20. The method of claim 13, wherein the administering increases an NAD⁺ level in the subject.
